(19) United States
(12) Patent Application Publication
(10) Pub. No.: US 2026/0000849 A1
(43) Pub. Date: Jan. 1, 2026

(54) INSUFFLATOR AND A METHOD FOR INSUFFLATING A CAVITY IN THE BODY OF A HUMAN OR ANIMAL SUBJECT AND AN INSUFFLATING METHOD AND SYSTEM FOR REMOTELY INSUFFLATING A CAVITY IN THE BODY OF A HUMAN OR ANIMAL SUBJECT

(71) Applicant: Palliare Limited, Dangan (IE)

(72) Inventor: John O'DEA, Bearna (IE)

(73) Assignee: Palliare Limited, Dangan (IE)

(21) Appl. No.: 19/316,385

(22) Filed: Sep. 2, 2025

Related U.S. Application Data
(63) Continuation-in-part of application No. 18/635,459, filed on Apr. 15, 2024.
Application No. 18/635,459 is a continuation-in-part of application No. 18/699,921, filed on Apr. 10, 2024.

(30) Foreign Application Priority Data
Jan. 12, 2022 (IE) S2022/0005
Apr. 14, 2023 (IE) S2023/0096

(51) Int. Cl. A61M 13/00

(52) U.S. Cl. CPC A61M 13/003; A61M 2202/0007; A61M 2202/02; A61M 2205/3344; A61M 2205/3379; A61M 2205/3553; A61M 2205/3592; A61M 2205/502; A61M 2205/583; A61M 2210/1021

(57) ABSTRACT

An insufflator is operable in a set-up mode and in a normal insufflating run mode. In the set-up mode, a pressure/volume relationship between cavity pressure and cavity volume of the cavity of a subject is determined. A minimum safe working pressure value and a maximum safe working pressure value of a working pressure range for the cavity to be insufflated is determined from the pressure/volume relationship as a pressure value just above a lower point of inflection and an upper point of inflection of the pressure/volume relationship, respectively. In the normal insufflating run mode a working pressure value is selected from the range of working pressure values and the insufflator insufflates the cavity to the selected working pressure value. Also disclosed is an insufflating system comprising an insufflator operable in a set-up mode and in a normal insufflating run mode. A controller located remotely relative to the insufflator remotely controls the insufflator to operate in the set-up mode and/or the normal insufflating run mode.

### CROSS-REFERENCE TO RELATED APPLICATIONS

This is application is a Continuation-In-Part of U.S. application Ser. No. 18/635,459, filed Apr. 15, 2024, which is a Continuation-In-Part of U.S. application Ser. No. 18/699,921, filed Apr. 10, 2024, which is a National Stage of International Application No. PCT/IE2023/000001 filed Jan. 12, 2023, claiming priority based on Irish Patent Application No. S2022/0005 filed Jan. 12, 2022. This application also claims priority based on Irish Patent Application No. S2023/0096 filed Apr. 14, 2023.

The present invention relates to an insufflator for insufflating a cavity in the body of a human or animal subject, and in particular, though not limited to the peritoneal cavity of a subject. The invention also relates to a method for insufflating a cavity in the body of a human or animal subject, and additionally, the invention relates to a method for determining a working pressure range for insufflating a cavity in the body of a human or animal subject. The invention also relates to an insufflating method and an insufflating system for remotely insufflating a cavity, such as a peritoneal cavity in a human or animal subject.

Medical insufflators are used in minimally invasive procedures, such as surgical or investigative procedures to create a pneumoperitoneum, namely, a working volume that allows a surgeon to operate laparoscopically and endoscopically in the peritoneal cavity or other cavities in the body of a human or animal subject, thereby providing a working volume therein for visualisation and manipulation of surgical tools and instruments. Current uses of insufflators require that surgeons or clinicians select an insufflating pressure based on heuristic methods, for example, quoted from clinical literature. As insufflating gas is being delivered to the peritoneal or other cavity pressure increases within the cavity, and the cavity expands creating a working volume for the surgeon. However, the pressure required to achieve an adequate working volume varies, depending on the compliance of the relevant cavity. For example, in the case of a heavy subject with a lot of body fat, the cavity in general will not expand to the same extent as a corresponding cavity of a light subject with little or no body fat, therefore requiring a higher insufflating pressure than that normally quoted in the literature. Conversely, commonly quoted insufflating pressure values in the literature may be inappropriately high for a lighter, thinner subject with little or no body fat. In other words, adequate working volume in the peritoneal or other cavity could be achieved at a lower pressure in a lighter person than in a heavier person. Additionally, the position of a subject on an operating table may also influence the insufflating pressure required to gain an adequate working volume, and in turn visualisation, in the peritoneal or other cavity, for example, depending on the position of the subject on the operating table, the legs of a subject may squeeze the abdomen, thereby reducing the compliance of, for example, the peritoneal cavity.

Accordingly, based on heuristic methods, the peritoneal cavity, and other cavities in one subject may be insufflated to a pressure in excess of a pressure required for insufflating a similar cavity in another subject. In other cases, the peritoneal cavity and other cavities, may be insufflated in a subject to a pressure where the cavity is becoming non-compliant to the extent that the delivery of further insufflating gas to the cavity would result in a significant increase in pressure in the cavity with little or no gain in the working volume of the cavity. It is desirable that during inflating of a cavity in a human or animal subject, the pressure in the cavity should be maintained at the lowest possible pressure consistent with providing an adequate working volume therein, in order to avoid any clinical risks, such as post operative pain, reduction in venous return, and other such complications which may arise when a cavity is insufflated to a pressure beyond a safe insufflating pressure.

It is also common practice during initial insufflating of a peritoneal cavity in a subject for the surgeon to tap the abdominal wall of the subject to ascertain the degree to which the abdominal wall is stretched due to the cavity pressure in the peritoneal cavity. If the tap on the abdominal wall of the subject should indicate that the abdominal wall is stretched taut, the surgeon would take that as an indication that the peritoneal cavity had been adequately insufflated. However, tapping the abdominal wall of a subject does not give a reliable indication that the peritoneal cavity has been insufflated to a suitable working pressure.

Furthermore, it is now common for minimally invasive procedures be they investigative or surgical procedures to be carried out remotely where the surgeon is not in the operating theatre where the procedure is being carried out on the subject, and in many cases, the surgeon may be many kilometres from the subject. Thus, in such cases, it is impossible for a surgeon to tap the abdominal wall of a subject as has been the practice heretofore, where such minimally invasive procedures had been carried out with the surgeon in the operating theatre directly operating the instruments to carry out the minimally invasive procedure. Furthermore, as discussed above, in carrying out minimally invasive procedures, it is important that the pressure in the cavity being insufflated is maintained at the lowest possible pressure consistent with adequate visualisation in the cavity. To some extent this is possible in such minimally invasive procedures being carried out by a surgeon who is present in the theatre in which the procedure is being carried out, since such a surgeon has direct access to the insufflator, and can control the insufflator as the procedure progresses. Also, in such minimally invasive procedures, a surgeon who is carrying out the procedure may require the pressure in the cavity being insufflated to be reduced, for example, in order to grasp a portion of the wall of the cavity being insufflated, which may include an anomaly, and in other cases, a surgeon may require the cavity pressure to be increased in order to increase visualisation for detecting relatively small anomalies which are difficult to visually detect. While in such minimally invasive procedures which are being carried out by a surgeon who is present in the theatre where the procedure is being carried out, the altering of the cavity pressure either to reduce the cavity pressure or to increase the cavity pressure, in general does not present a problem, since the surgeon has direct access to the insufflator. However in the carrying out of such minimally invasive procedures remotely, where a surgeon is in location remote from the location in which the procedure is being carried out, such alterations in pressure and also maintaining the pressure in the cavity at the lowest possible pressure consistent with adequate visualisation is in general not possible, since the surgeon no longer has direct access to the insufflator.

Accordingly, it would be advantageous for a surgeon or clinician to be aware of the working pressure range of a cavity in the body of a human or animal subject prior to insufflating the cavity during the carrying out of a minimally invasive investigative or surgical procedure, and it would also be advantageous for a surgeon or a clinician carrying out a minimally invasive procedure in a location remote from the location in which the procedure is being carried out to be able to maintain the cavity pressure at the lowest possible pressure consistent with adequate visualisation in the cavity, and also to be in a position to alter the cavity pressure more readily.

The present invention is directed towards providing an insufflator for insufflating a cavity in the body of a human or animal subject which is adapted for determining the working pressure range of a cavity in the body of a human or animal subject. The invention is also directed towards a method for insufflating a cavity in the body of a human or animal subject which provides for determining the working pressure range of the cavity of the subject. Further, the invention is directed towards providing a method for determining the working pressure range for a cavity in the body of a human or animal subject. The invention is also directed towards an insufflating method and an insufflating system for remotely insufflating a cavity in a human or animal subject.

According to the invention there is provided an insufflator adapted to be selectively operated in a normal insufflating mode and in a set-up mode, the insufflator being configured in the set-up mode for determining the value of an optimum maximum pressure for insufflating a cavity in the body of a human or animal subject, the insufflator comprising:
a delivery means for delivering insufflating gas to the cavity,
a pressure sensor for producing a signal indicative of the pressure in the cavity,
a flow sensor for monitoring flow of insufflating gas being delivered to the cavity and for producing a signal indicative of the cumulative volume of insufflating gas delivered to the cavity from the commencement of delivery of the insufflating gas thereto or a signal indicative of the rate at which the insufflating gas is being delivered to the cavity, and
a signal processor adapted to read the signal produced by the pressure sensor, and to read the signal produced by the flow sensor during insufflating of the cavity, and in the set-up mode:
   to determine a pressure/volume relationship between the pressure in the cavity and insufflating gas delivered to the cavity as the insufflating gas is being delivered to the cavity from values of the signals read from the pressure sensor and the flow sensor, and
to determine the value of the optimum maximum pressure as the value of a transition pressure at which the pressure/volume relationship transitions from a first pressure/volume relationship to a second pressure/volume relationship, the second pressure/volume relationship being different to the first pressure/volume relationship.

In one embodiment of the invention the pressure/volume relationship determined by the signal processor comprises the value of the increase in pressure in the cavity per unit volume of insufflating gas delivered to the cavity.

In another embodiment of the invention the first pressure/volume relationship comprises either a substantially linear relationship or a non-linear relationship, and preferably, the first pressure/volume relationship comprises a substantially linear relationship during which the pressure in the cavity increases linearly with respect to the delivery of insufflating gas to the cavity, and in another embodiment of the invention the first pressure/volume relationship comprises a substantially linear relationship during which the increase in pressure in the cavity per unit volume of insufflating gas delivered to the cavity remains at a substantially constant value, and preferably, a substantially constant value greater than zero.

In another embodiment of the invention the second pressure/volume relationship comprises either a substantially linear relationship or a non-linear relationship.

In another embodiment of the invention the second pressure/volume relationship comprises a substantially linear relationship during which the pressure in the cavity increases linearly with respect to the delivery of insufflating gas to the cavity, and preferably, the second pressure/volume relationship comprises a substantially linear relationship during which the increase in pressure in the cavity per unit volume of insufflating gas delivered to the cavity remains at a substantially constant value, and in one embodiment of the invention the value of the increase in the pressure in the cavity per unit volume of insufflating gas delivered to the cavity in the second pressure/volume relationship, is greater than the value of the increase in the pressure in the cavity per unit volume of insufflating gas delivered to the cavity in the first pressure/volume relationship.

In another embodiment of the invention the first pressure/volume relationship transitions to the second pressure/volume relationship through an intermediate pressure/volume relationship, and preferably, the intermediate pressure/volume relationship comprises a non-linear relationship.

In one embodiment of the invention the signal processor is programmed to determine the transition pressure value as a pressure value lying in a range between a first pressure value and a second pressure value, and the signal processor is programmed to determine the first pressure value as the pressure at which the first pressure/volume relationship transitions to the intermediate pressure/volume relationship, and the signal processor is programmed to determine the second pressure value as the pressure at which the intermediate pressure/volume relationship transitions to the second pressure/volume relationship.

In one embodiment of the invention the signal processor is programmed to determine the transition pressure value as the average value of the first and the second pressure values.

In another embodiment of the invention the signal processor is programmed to determine the transition pressure value as a point of inflection on a line representative of a graph of the pressure/volume relationship during insufflating of the cavity, as the first pressure/volume relationship transitions to the second pressure/volume relationship.

In another embodiment of the invention in the graph of the pressure/volume relationship, volume is plotted on the abscissa, and pressure is plotted on the ordinate of the graph.

Preferably, the signal processor is programmed to determine the point of inflection by interpolating the point of intersection of a portion of the line representative of the first pressure/volume relationship and a portion of the line representative of the second pressure/volume relationship. Alternatively, the signal processor is programmed to determine the point of inflection on the line of the graph representative of the pressure/volume relationship during insufflating of the cavity by extrapolating the portion of the line representing the first pressure/volume relationship beyond the first pressure value, and extrapolating the line representing the second pressure/volume relationship beyond the second pressure value, and to determine the value of the transition pressure at the point of intersection of the extrapolated parts of the lines representing the first and second pressure/volume relationship.

In another embodiment of the invention the signal processor is programmed to read the values of the signals produced by the pressure sensor and the flow sensor either continuously or at predefined time intervals. Preferably, the signal processor is programmed to time-stamp, cross-reference and store in memory each pair of the values of the signals read from the pressure sensor and the flow sensor.

In one embodiment of the invention the signal processor is programmed to determine the value of the transition pressure from the stored, time-stamped and cross-referenced values of the pairs of values of the signals read from the pressure sensor and the flow sensor.

In another embodiment of the invention the signal processor is programmed to determine the value of the cumulative volume of insufflating gas delivered to the cavity and the corresponding value of the pressure in the cavity, each time the values of the signals are read from the flow sensor and the pressure sensor, and to time-stamp, cross-reference and store in memory each pair of the determined value of the cumulative volume of the insufflating gas delivered to the cavity and the corresponding value of the pressure in the cavity. Preferably, the signal processor is programmed to determine the value of the transition pressure from the stored, cross-referenced and time-stamped pairs of values of the cumulative volume of insufflating gas delivered to the cavity and the corresponding pressure in the cavity.

In one embodiment of the invention the signal processor is programmed to compute the value of the increase in the pressure in the cavity per unit volume of insufflating gas delivered to the cavity each time the values of the signals are read by the signal processor from the pressure sensor and the flow sensor. Preferably, the signal processor is programmed to apply a smoothing algorithm to the computation of each value of the increase in pressure in the cavity per unit volume of insufflating gas delivered to the cavity, and preferably, the smoothing algorithm comprises a moving average algorithm. Advantageously, the signal processor is programmed to time-stamp, cross-reference and store in memory each computed value of the increase in the pressure in the cavity per unit volume of insufflating gas delivered to the cavity and the corresponding value of the pressure in the cavity.

In one embodiment of the invention the signal processor is programmed to determine the value of the transition pressure from the computed values of the increase in pressure in the cavity per unit volume of insufflating gas delivered to the cavity and the corresponding values of the pressure in the cavity.

In one embodiment of the invention the signal processor is programmed to determine the first pressure value from the computed values of the increase in the pressure in the cavity per unit volume of the insufflating gas delivered to the cavity and the corresponding values of the pressure in the cavity.

In another embodiment of the invention the signal processor is programmed to determine the second pressure value from the computed values of the increase in the pressure in the cavity per unit volume of the insufflating gas delivered to the cavity and the corresponding values of the pressure in the cavity and the corresponding values of the pressure in the cavity.

In another embodiment of the invention the signal processor is programmed to store the value of the optimum maximum pressure in memory.

Preferably, the signal processor is programmed to produce a signal indicative of the value of the optimum maximum pressure. Preferably, the signal produced by the signal processor indicative of the value of the optimum maximum pressure is adapted for conversion to a human sensory perceptible signal.

Advantageously, the signal indicative of the value of the optimum maximum pressure produced by the signal processor is adapted for applying to a visual display screen for displaying the value of the optimum maximum pressure thereon.

In one embodiment of the invention the signal processor is programmed to terminate delivery of insufflating gas to the cavity in the set-up mode in response to the pressure in the cavity reaching a pressure beyond which the cavity can no longer be safely insufflated.

In another embodiment of the invention the signal processor is programmed to terminate delivery of insufflating gas to the cavity in the set-up mode in response to the cavity pressure reaching a maximum set-up safe pressure.

In another embodiment of the invention the maximum set-up safe pressure is either selectable or predefined.

In one embodiment of the invention the maximum set-up safe pressure lies in the range of 20 mmHg to 25 mmHg. Preferably, the maximum set-up safe pressure lies in the range of 10 mmHg to 15 mmHg. Advantageously, the maximum set-up safe pressure is approximately 15 mmHg.

In another embodiment of the invention the signal processor is programmed to terminate delivery of insufflating gas to the cavity in the set-up mode in response to the value of the transition pressure being determined.

In another embodiment of the invention the signal processor is programmed to terminate delivery of insufflating gas to the cavity in the set-up mode in response to the second pressure value being determined.

In one embodiment of the invention the signal processor is programmed to limit the supply of insufflating gas to the cavity in response to the pressure in the cavity reaching the optimum maximum pressure value when the insufflator is operating in the normal insufflating mode.

In another embodiment of the invention the signal processor is programmed to terminate the supply of insufflating gas to the cavity in response to the pressure in the cavity exceeding the optimum maximum pressure when the insufflator is operating in the normal insufflating mode.

In another embodiment of the invention the signal processor is programmed to terminate the supply of insufflating gas to the cavity in response to the pressure in the cavity reaching the optimum maximum pressure value when the insufflator is operating in the normal insufflating mode.

In one embodiment of the invention the signal processor is programmed to reinstate the supply of insufflating gas to the cavity in response to the pressure in the cavity falling below the optimum maximum pressure value when the insufflator is operating in the normal insufflating mode.

The invention also provides a method for determining an optimum maximum pressure value for insufflating a cavity in the body of a human or animal subject, the method comprising:
delivering insufflating gas to the cavity,
determining a pressure/volume relationship between the pressure in the cavity and insufflating gas delivered to the cavity as the insufflating gas is being delivered to the cavity,
determining the optimum maximum pressure value as the value of a transition pressure at which the pressure/volume relationship transitions from a first pressure/volume relationship to a second pressure/volume relationship, the second pressure/volume relationship being different to the first pressure/volume relationship.

In one embodiment of the invention the determined pressure/volume relationship comprises the value of the increase in pressure in the cavity per unit volume of insufflating gas delivered to the cavity.

In one embodiment of the invention the first pressure/volume relationship comprises either a substantially linear relationship or a non-linear relationship, and preferably, the first pressure/volume relationship comprises a substantially linear relationship during which the pressure in the cavity increases with respect to the delivery of insufflating gas to the cavity, and in another embodiment of the invention the first pressure/volume relationship comprises a substantially linear relationship during which the value of the increase in the pressure in the cavity per unit volume of insufflating gas delivered to the cavity remains at a substantially constant value, and preferably, at a substantially constant value greater than zero.

In another embodiment of the invention the second pressure/volume relationship comprises either a substantially linear relationship or a non-linear relationship.

In another embodiment of the invention the second pressure/volume relationship comprises a substantially linear relationship during which the pressure in the cavity increases with respect to the delivery of insufflating gas to the cavity, and preferably, the second pressure/volume relationship comprises a substantially linear relationship, during which the value of the increase in pressure in the cavity per unit volume of insufflating gas delivered to the cavity remains at a substantially constant value, and preferably, at a substantially constant value greater than the substantially constant value of the increase in pressure in the cavity per unit volume of insufflating gas delivered to the cavity during the first pressure/volume relationship.

In another embodiment of the invention the first pressure/volume relationship transitions to the second pressure/volume relationship through an intermediate pressure/volume relationship, and preferably, the intermediate pressure/volume relationship comprises a non-linear relationship.

In another embodiment of the invention the transition pressure value is determined as a pressure value lying in a range between a first pressure value and a second pressure value, the first pressure value being determined as the pressure at which the first pressure/volume relationship transitions to the intermediate pressure/volume relationship, and the second pressure value being determined as the pressure at which the intermediate pressure/volume relationship transitions to the second pressure/volume relationship.

In one embodiment of the invention the transition pressure value is determined as the average value of the first and second pressure values.

In another embodiment of the invention the transition pressure value is determined as a point of inflection on a line of a graph representative of the pressure/volume relationship during insufflating of the cavity as the first pressure/volume relationship transitions to the second pressure/volume relationship.

In another embodiment of the invention in the graph representative of the pressure/volume relationship, volume is plotted on the abscissa, and pressure is plotted on the ordinate.

In one embodiment of the invention the point of inflection is determined by interpolating the point of intersection of a portion of the line representative of the first pressure/volume relationship and a portion of the line representative of the second pressure/volume relationship. In an alternative embodiment of the invention the portion of the line representative of the first pressure/volume relationship is extrapolated beyond the first pressure value, and the portion of the line representative of the second pressure/volume relationship is extrapolated beyond the second pressure value, and the point of inflection is determined as the point of intersection of the extrapolated portion of the line representative of the first pressure/volume relationship and the extrapolated portion of the line representative of the second pressure/volume relationship.

In one embodiment of the invention the value of the pressure in the cavity and the corresponding value of either the rate at which insufflating gas is being delivered to the cavity or the cumulative volume of insufflating gas delivered to the cavity are determined either continuously or at predefined time intervals.

In another embodiment of the invention each pair of the determined values of either the pressure in the cavity and the corresponding rate at which the insufflating gas is being delivered to the cavity, or the pressure in the cavity and the corresponding cumulative volume of insufflating gas delivered to the cavity are time-stamped, cross-referenced and stored.

In one embodiment of the invention the value of the transition pressure is determined from the pairs of the values of the pressure in the cavity and the corresponding rate at which insufflating gas is being delivered to the cavity.

In another embodiment of the invention the value of the transition pressure is determined from the stored, cross-referenced and time-stamped pairs of values of the pressure in the cavity and the corresponding cumulative volume of insufflating gas delivered to the cavity.

Preferably, the value of the increase in the pressure of the cavity per unit volume of insufflating gas delivered to the cavity is computed from each pair of the determined values of the pressure in the cavity and the corresponding rate of delivery of insufflating gas to the cavity, or from each pair of the determined values of the pressure in the cavity and the corresponding cumulative volume of insufflating gas delivered to the cavity. Preferably, a smoothing algorithm is applied to each computation of the increase in pressure in the cavity per unit volume of insufflating gas delivered to the cavity, and preferably, the smoothing algorithm comprises a moving average algorithm. Advantageously, each computed value of the increase in pressure in the cavity per unit increase in the volume of insufflating gas delivered to the cavity and the corresponding value of the pressure in the cavity are time-stamped, cross-referenced and stored.

In one embodiment of the invention the value of the transition pressure is determined from the computed values of the increase in pressure in the cavity per unit volume of insufflating gas delivered to the cavity and the corresponding values of the pressure in the cavity.

In another embodiment of the invention the first pressure value is determined from the computed values of the increase in the pressure in the cavity per unit volume of insufflating gas delivered to the cavity and the corresponding values of the pressure in the cavity.

In another embodiment of the invention the second pressure value is determined from the computed values of the increase in the pressure in the cavity per unit volume of insufflating gas delivered to the cavity and the corresponding values of the pressure in the cavity.

Preferably, the pressure/volume relationship is determined each time the value of the pressure in the cavity and the corresponding value of either the rate at which the insufflating gas is being delivered to the cavity or the cumulative volume of insufflating gas delivered to the cavity are determined.

In another embodiment of the invention delivery of insufflating gas to the cavity is terminated in response to the pressure in the cavity reaching a maximum safe pressure value beyond which the cavity can no longer be safely insufflated.

In one embodiment of the invention the maximum safe pressure value is selectable or predefined.

In another embodiment of the invention the maximum safe pressure value lies in the range of 25 mmHg to 30 mmHg. Advantageously, the maximum safe pressure value is approximately 30 mmHg.

In another embodiment of the invention delivery of insufflating gas to the cavity is terminated in response to the value of the transition pressure being determined.

In another embodiment of the invention delivery of insufflating gas to the cavity is terminated in response to the second pressure value being determined.

Preferably, the insufflating gas is delivered to the cavity at a relatively slow rate, while the value of the optimum maximum pressure is being determined.

In one embodiment of the invention a signal indicative of the value of the optimum maximum pressure is produced. Preferably, the signal indicative of the value of the optimum maximum pressure comprises a human sensory perceptible signal.

Advantageously, the signal indicative of the value of the optimum maximum pressure is adapted for applying to a visual display screen, for display thereon. Preferably, the signal indicative of the value of the optimum maximum pressure is adapted for storing in an electronic memory of an insufflator.

Additionally, the invention provides a method for operating an insufflator for insufflating a cavity in the body of a human or animal subject, the insufflator comprising a delivery means for delivering insufflating gas to the cavity, and a signal processor for controlling the operation of the delivery means, the method comprising storing the value of the optimum maximum pressure value determined by the method according to the invention in an electronic memory of the insufflator, and programming the signal processor to control the delivery means to limit the delivery of insufflating gas to the cavity or to produce a signal adapted for applying to a means for producing a human sensory perceptible signal warning of the pressure in the cavity reaching the optimum maximum pressure, in response to the pressure in the cavity reaching the optimum maximum pressure value.

In one embodiment of the invention the signal processor is programmed to terminate delivery of insufflating gas to the cavity in response to the pressure in the cavity reaching the optimum maximum pressure value.

In another embodiment of the invention the delivery of insufflating gas to the cavity is reinstated on the pressure in the cavity falling below the optimum maximum pressure value.

In a further embodiment of the invention the insufflator comprises a pressure sensor for monitoring the pressure in the cavity, the pressure sensor being configured to produce a signal indicative of the pressure in the cavity, and the signal processor is programmed to control the delivery means to maintain the pressure in the cavity substantially at a selectable desired working pressure in response to the value of the signal indictive of the pressure in the cavity read from the pressure sensor.

The invention also provides an insufflator selectively operable in an insufflating mode for insufflating a cavity in the body of a human or animal subject and in a set-up mode for determining a working pressure range for the cavity, the insufflator comprising:
a flow control means adapted for controlling flow of insufflating gas for insufflating the cavity,
a pressure sensor adapted to monitor pressure in the cavity (cavity pressure) and to produce a signal indicative of the cavity pressure,
a flow sensor adapted to monitor flow of insufflating gas to the cavity and to produce a signal indicative of flow of insufflating gas to the cavity,
a signal processor, and
an electronic storing means accessible to the signal processor,
the signal processor being programmed
   to read the signals from the pressure sensor and from the flow sensor at predefined time intervals during insufflating of the cavity in the set-up mode,
   to determine a pressure/volume relationship between cavity pressure and the volume of the cavity from the signals read from the pressure sensor and the flow sensor,
   to determine a minimum working pressure value of the working pressure range below which cavity pressure should not fall, and an optimum maximum pressure value of the working pressure range above which the cavity pressure should not exceed from the pressure/volume relationship between the cavity pressure and the volume of the cavity, and
   to store the minimum working pressure value and the optimum maximum pressure value in the storing means.

In one embodiment of the invention the signal processor is programmed to determine the minimum working pressure value as the cavity pressure at which the cavity pressure commences to increase after commencement of insufflating of the cavity in the set-up mode.

In another embodiment of the invention the signal processor is programmed to determine the minimum working pressure value as the cavity pressure at a first point of inflection of a graph representative of the pressure/volume relationship between the cavity pressure and the volume of the cavity.

Preferably, the signal processor is programmed to determine the minimum working pressure value as the cavity pressure at which an initial pressure/volume relationship of the pressure/volume relationship between the cavity pressure and the volume of the cavity during which the cavity pressure remains substantially constant transitions to a first pressure/volume relationship during which the increase in cavity pressure per unit volume of insufflating gas delivered to the cavity is substantially constant.

In one embodiment of the invention the signal processor is programmed to determine the optimum maximum pressure value as the cavity pressure at which the increase in the volume of the cavity per unit increase in cavity pressure commences to decrease or is minimal, or as the cavity pressure at a second point of inflection of a graph representative of the pressure/volume relationship.

Advantageously, the signal processor is programmed to determine the optimum maximum pressure value as the cavity pressure at which the pressure/volume relationship between the cavity pressure and the volume of the cavity transitions from the first pressure/volume relationship during which the increase in cavity pressure per unit volume of insufflating gas delivered to the cavity is substantially constant to a second pressure/volume relationship during which the increase in cavity pressure per unit volume of insufflating gas delivered to the cavity is substantially constant, but is greater than the increase in cavity pressure per unit volume of insufflating gas delivered to the cavity during the first pressure/volume relationship.

In a further embodiment of the invention the flow control means is operable under the control of the signal processor in the set-up mode for delivering the insufflating gas to the cavity at a substantially constant rate.

Preferably, the signal processor is programmed in the set-up mode to cease insufflating of the cavity in response to the cavity pressure reaching a maximum safe pressure value, and preferably, the maximum safe pressure value is greater than the optimum maximum pressure value.

In one embodiment of the invention the signal processor is programmed to output a signal indicative of the minimum working pressure value and the optimum maximum pressure value to a visual display screen for displaying thereon.

In another embodiment of the invention the signal processor is programmed to determine a plurality of intervening pressure values between the minimum working pressure value and the optimum maximum pressure value of the working pressure range, and to store the intervening pressure values in the electronic storing means along with the minimum working pressure value and the optimum maximum pressure value in the electronic storing means as the working pressure range. Preferably, the signal processor is programmed to output a signal indicative of the working pressure range to an interface means configured to enable selection of a working pressure value from the working pressure range through the interface means.

In another embodiment of the invention the signal processor is programmed to determine a minimum working volume value of a working volume range for the cavity corresponding to the minimum working pressure value, and to determine a maximum working volume value of the working volume range for the cavity corresponding to the optimum maximum pressure value thereof and to store the minimum working volume value and the maximum working volume value in the electronic storing means.

In another embodiment of the invention the signal processor is programmed to determine a plurality of intervening volume values of the cavity corresponding to the intervening pressure values between the minimum working volume value and the maximum working volume value, and to store the intervening working volume values for the cavity along with the minimum working volume value and the maximum working volume value as the working volume range in the electronic storing means.

In another embodiment of the invention the signal processor is programmed to store the minimum working volume value, the maximum working volume value and the intervening volume values in the form of a look-up table cross-referenced with the corresponding minimum working pressure value, the optimum maximum pressure value and the intervening pressure values in the electronic storing means.

In another embodiment of the invention the signal processor is programmed to output a signal indicative of the minimum and maximum working volume values and the intervening volume values of the working volume range to the interface means to enable a selection of a working volume value from the working volume range through the interface means.

In an alternative embodiment of the invention an image of a graphical representation of the first pressure/volume relationship of the cavity between the minimum working pressure value and the optimum maximum pressure value is displayed on an interface means to enable selection of the working pressure value or the working volume value at which the cavity is to be insufflated to be selected through the interface means.

In one embodiment of the invention the graphical representation of the first pressure/volume relationship is displayed on a visual display screen of the interface means, and preferably, the interface means comprises a touch screen, and preferably, the graphical representation of the first pressure/volume relationship between the minimum working pressure value and the optimum maximum pressure value is displayed on the touch screen, and the working pressure value or the working volume value at which the cavity is to be insufflated, is selectable by touching the graphical representation at a point thereof indicative of the cavity pressure and the cavity volume corresponding to the working pressure value and the working volume value at which the cavity is to be insufflated.

In one embodiment of the invention the graphical representation of the first pressure/volume relationship between the minimum working pressure value and the optimum maximum pressure value is displayed on a grid representation with pressure values of cavity pressure on an ordinate axis or an abscissa axis and the corresponding values of the volume of the cavity on the other one of the ordinate axis and the abscissa axis. Preferably, the cavity pressure values are identified on the ordinate axis and the corresponding volume values of the cavity are identified on the abscissa axis.

In one embodiment of the invention the volume values are identified as proportions of the maximum volume value corresponding to the optimum maximum pressure value. Advantageously, the cavity pressure values are identified as actual pressure values.

In one embodiment of the invention the selectable values of the working pressure values are infinitely selectable from the minimum working pressure value to the optimum maximum pressure value.

In a further embodiment of the invention the current cavity pressure is identified on the graphical representation of the first pressure/volume relationship by a first indicating means.

In one embodiment of the invention the first indicating means comprises a first cursor, and preferably, the first cursor is configured to move along the graphical representation of the first pressure/volume relationship in response to change in the cavity pressure, and preferably, in response to the signal read from the pressure sensor indicative of the cavity pressure.

In another embodiment of the invention a second indicating means is provided for identifying the selected working pressure value or the working volume value at which the cavity is to be insufflated. Preferably, the second indicating means comprises a second cursor, and advantageously, the second cursor is moveable along the graphical representation of the first pressure/volume relationship to the cavity pressure or the cavity volume corresponding to the working pressure value or the working volume value at which the cavity is to be insufflated.

In one embodiment of the invention the second cursor is moveable along the graphical representation of the first pressure/volume relationship by touching the second cursor on the touch screen.

In another embodiment of the invention the second cursor is moveable along the graphical representation by an input means, and preferably, by an input signal generated by the input means, which may comprise a manually or a foot pedal operated switch.

In another embodiment of the invention the pressure/volume relationship including the initial pressure/volume relationship, the first pressure/volume relationship and the second pressure/volume relationship are displayed on the interface means.

In one embodiment of the invention the graphical representation of the pressure/volume relationship or the first pressure/volume relationship displayed on the interface means comprises a smoothed version of the pressure/volume relationship or the first pressure/volume relationship.

In one embodiment of the invention the flow control means is adapted for delivering insufflating gas to the cavity and for drawing insufflating gas from the cavity.

In another embodiment of the invention the flow control means comprises a flow controller operable under the control of the signal processor in the set-up mode and in the insufflating mode for delivering insufflating gas to the cavity.

In another embodiment of the invention the signal processor in the insufflating mode of the insufflator is responsive to a signal indicative of a selected working pressure value of the working pressure range for controlling the flow controller in response to the signal from the pressure sensor indicative of the cavity pressure for maintaining the cavity pressure at the selected working pressure value.

In a further embodiment of the invention the flow control means comprises a vacuum applying means operable under the control of the signal processor for applying a vacuum to the cavity for drawing insufflating gas or smoke from the cavity.

In another embodiment of the invention the signal processor is responsive to the cavity pressure exceeding the selected working pressure value for operating the vacuum applying means for applying a vacuum to the cavity to reduce the pressure in the cavity to the selected working pressure value.

In another embodiment of the invention the signal processor is responsive to a signal indicative of a selected working pressure value greater than the working pressure value at which the cavity is being insufflated for operating the flow controller to increase the flow of insufflating gas to the cavity to increase the cavity pressure to the selected working pressure value.

In a further embodiment of the invention the signal processor is responsive to a signal indicative of a selected working pressure value less than the working pressure value at which the cavity is being insufflated for operating the vacuum applying means in response to the signal from the pressure sensor indicative of the cavity pressure to apply a vacuum to the cavity for reducing the cavity pressure to the selected working pressure value.

In another embodiment of the invention the signal processor is responsive to an externally generated signal indicative of a selected working pressure value to which the cavity pressure is to be increased or decreased from the current working pressure value at which the cavity is being insufflated to operate the flow controller and/or the vacuum applying means in response to the signal from the pressure sensor indicative of the cavity pressure for increasing or decreasing the cavity pressure to the selected working pressure value.

In one embodiment of the invention the externally generated signal is produced from a foot pedal operated switch, and preferably, from two foot pedal operated switches, one of the foot pedal operated switches being configured to produce the externally generated signal indicative of an increase in the cavity pressure, and the other one of the foot pedal operated switches being configured to produce the externally generated signal indicative of a decrease in the cavity pressure.

Further the invention provides a method for determining a minimum working pressure value and an optimum maximum pressure value of a working pressure range for insufflating a cavity in the body of a human or animal subject, the method comprising:
delivering insufflating gas to the cavity,
monitoring cavity pressure values and corresponding values of cumulative volume of insufflating gas delivered to the cavity at predefined time intervals from the commencement of delivery of insufflating gas to the cavity,
determining a pressure/volume relationship between the cavity pressure and the volume of the cavity from the monitored cavity pressure values and the monitored cumulative volume values, and
determining the minimum working pressure value and the optimum maximum pressure value of the working pressure range from the pressure/volume relationship.

In one embodiment of the invention the minimum working pressure value is determined as the cavity pressure at which the cavity pressure commences to rise after commencement of insufflating of the cavity.

In another embodiment of the invention the minimum working pressure value is determined as the cavity pressure at a first point of inflection of a graph representative of the pressure/volume relationship between the cavity pressure and the volume of the cavity.

In another embodiment of the invention the minimum working pressure value is determined as the cavity pressure at which an initial pressure/volume relationship of the pressure/volume relationship between the cavity pressure and the volume of the cavity during which the cavity pressure remains substantially constant transitions to a first pressure/volume relationship during which the increase in cavity pressure per unit volume of insufflating gas delivered to the cavity is substantially constant.

In another embodiment of the invention the optimum maximum pressure value is determined as the cavity pressure at which the increase in the volume of the cavity per unit increase in cavity pressure commences to decrease or is minimal, or as the cavity pressure at a second point of inflection of a graph representative of the pressure/volume relationship.

In another embodiment of the invention the optimum maximum pressure value is determined as the cavity pressure at which the pressure/volume relationship between the cavity pressure and the volume of the cavity transitions from the first pressure/volume relationship during which the increase in cavity pressure per unit volume of insufflating gas delivered to the cavity is substantially constant to a second pressure/volume relationship during which the increase in cavity pressure per unit volume of insufflating gas delivered to the cavity is substantially constant, but is greater than the increase in cavity pressure per unit volume of insufflating gas delivered to the cavity during the first pressure/volume relationship.

Preferably, the insufflating gas is delivered to the cavity at a substantially constant rate during the determining of the working pressure range.

In one embodiment of the invention the minimum working pressure value and the optimum maximum pressure value are stored, and preferably, are displayed on a visual display screen.

In another embodiment of the invention a plurality of intervening pressure values between the minimum working pressure value and the optimum maximum pressure value of the working pressure range are determined. Preferably, the plurality of intervening pressure values along with the minimum working pressure value and the optimum maximum pressure value of the working pressure range are stored.

In another embodiment of the invention a minimum working volume value of a working volume range for the cavity corresponding to the minimum working pressure value is determined, and a maximum working volume value of the working volume range for the cavity corresponding to the optimum maximum pressure value thereof is determined.

Preferably, the minimum working volume value of the working pressure range and the maximum working volume value of the working volume range are stored.

In another embodiment of the invention a plurality of intervening working volume values for the cavity corresponding to the intervening pressure values between the minimum working volume value and the maximum working volume value are determined, and preferably, are stored along with the minimum working volume value and the maximum working volume value as the working volume range.

In another embodiment of the invention the minimum working volume value, the maximum working volume value and the intervening volume values are stored in the form of a look-up table cross-referenced with the corresponding minimum working pressure value, the optimum maximum pressure value and the intervening pressure values to enable a selection of a working volume value from the working volume range.

Additionally, the invention provides a method for insufflating a cavity in the body of a human or animal subject at a selectable working pressure value within the working pressure range determined in accordance with the method for determining the working pressure range according to the invention, the method for insufflating the cavity comprising:
delivering insufflating gas to the cavity,
monitoring the cavity pressure,
controlling the delivery of insufflating gas to and from the cavity for maintaining the cavity pressure at the selectable working pressure value in response to the cavity pressure, and
comparing the cavity pressure with the minimum working pressure value, and increasing the delivery of insufflating gas to the cavity in the event of the cavity pressure falling below the minimum working pressure value to return the cavity pressure to or above the minimum working pressure value.

Preferably, the cavity pressure is compared with the optimum maximum pressure value, and insufflating gas is drawn from the cavity in the event of the cavity pressure exceeding the optimum maximum pressure value to reduce the cavity pressure to or below the optimum maximum cavity pressure value.

Preferably, the insufflating of the cavity is terminated in response to the cavity pressure reaching a maximum safe pressure value, and preferably, the maximum safe pressure value is greater than the optimum maximum pressure value.

In one embodiment of the invention insufflating gas is delivered to the cavity in response to the cavity pressure for maintaining the cavity at a selected working pressure value.

In another embodiment of the invention the flow of insufflating gas to the cavity is increased to increase the cavity pressure to the selected working pressure value.

In a further embodiment of the invention a vacuum is applied to the cavity for drawing insufflating gas or smoke therefrom for reducing the cavity pressure to the selected working pressure value.

In a further embodiment of the invention insufflating gas is delivered to the cavity to return the cavity pressure to the minimum working pressure value in response to the cavity pressure falling below the minimum working pressure value.

In another embodiment of the invention insufflating gas is drawn from the cavity to reduce the cavity pressure to the optimum maximum pressure value in response to the cavity pressure exceeding the optimum maximum pressure value.

In one embodiment of the invention the interface means comprises a visual display screen and an input means configured to input a signal indicative of a selected working pressure value.

Additionally the invention provides a method for remotely insufflating a cavity in the body of a human or an animal subject,
the subject being in a first location,
an insufflator located in the first location and connected to the cavity of the subject for insufflating thereof, the insufflator comprising a first communicating means, and
a controller located in a second location remote from the first location for controlling operation of the insufflator, the controller comprising a second communicating means adapted to remotely communicate with the first communicating means of the insufflator, for communicating control signals from the controller to the insufflator for controlling the operation of the insufflator, the method comprising:
   operating the controller to transmit a signal indicative of a desired working pressure value at which the cavity of the subject is to be maintained during insufflating thereof for reception by the first communicating means of the insufflator, and
   operating the controller to transmit a signal for reception by the first communicating means of the insufflator to operate the insufflator in an insufflating mode to insufflate the cavity of the subject at the desired working pressure value, the desired working pressure value being a pressure value selectable from a range of pressure values between a first transition pressure value and a second transition pressure value,
   the first transition pressure value being a pressure value in a pressure/volume relationship between the pressure in the cavity (cavity pressure) and the volume of the cavity of the subject at which the pressure/volume relationship transitions from an initial pressure/volume relationship during which the cavity pressure remains substantially constant while insufflating gas is being delivered to the cavity, to a first pressure/volume relationship during which the cavity pressure increases per unit volume of insufflating gas delivered to the cavity, and
   the second transition pressure value being a pressure value in the pressure/volume relationship at which the first pressure/volume relationship transitions to a second pressure/volume relationship during which the cavity pressure increases per unit volume of insufflating gas delivered to the cavity at a greater rate than the cavity pressure increases per unit volume of insufflating gas delivered to the cavity during the first pressure/volume relationship.

In one embodiment of the invention the increase in the cavity pressure per unit volume of insufflating gas delivered to the cavity in the second pressure/volume relationship is at least twice the increase in the pressure per unit volume of insufflating gas delivered to the cavity in the first pressure/volume relationship.

In one embodiment of the invention the first and second locations are located in respective different buildings, in respective different towns or cities, or in respective different countries, and preferably, the first and second communicating means are adapted to communicate wirelessly, by wire and/or fibreoptically.

In another embodiment of the invention the first and second communicating means are adapted to communicate over the internet or by other suitable communicating means.

In one embodiment of the invention the first and second locations are located in respective different buildings.

In another embodiment of the invention the first and second locations are located in respective different towns or cities.

In a further embodiment of the invention the first and second locations are located in respective different countries.

In one embodiment of the invention the controller comprises an interface means, and the desired working pressure value is inputted to the controller through the interface means thereof for transmission by the controller to the insufflator.

Preferably, the interface means of the controller comprises a touch screen interface.

In another embodiment of the invention a pressure value to which the cavity pressure is to be altered from the current cavity pressure is inputted through the interface means of the controller, and the controller is adapted to transmit to the insufflator an alter cavity pressure request signal indicative of the pressure value to which the cavity pressure is to be altered from the current cavity pressure during insufflating of the cavity.

In another embodiment of the invention the interface means of the controller comprises at least one switch means, and the at least one switch means is adapted for inputting the pressure value to which the cavity pressure is to be altered from the current cavity pressure and for producing the alter cavity pressure request signal for transmission to the insufflator.

In another embodiment of the invention the interface means of the controller comprises a pair of the switch means, a first one of the switch means being adapted for inputting a pressure value to which the cavity pressure is to be increased from the current cavity pressure and for producing the alter cavity pressure request signal as an increase pressure request signal indicative of the pressure value to which the cavity pressure is to be increased from the current cavity pressure, and a second one of the switch means being adapted for inputting a pressure value to which the cavity pressure is to be reduced from the current cavity pressure and for producing the alter cavity pressure request signal as a decrease pressure request signal indicative of the pressure value to which the cavity pressure is to be reduced from the current cavity pressure.

In one embodiment of the invention each switch means of the controller comprises a foot pedal operated switch.

In one embodiment of the invention the first and second transition pressure values are visually displayed on the interface means of the controller.

In another embodiment of the invention the first pressure/volume relationship is visually displayed on the interface means of the controller.

In one embodiment of the invention the first pressure/volume relationship is visually displayed as a graphical representation on the interface means of the controller, and preferably, the pressure/volume relationship is visually displayed as a graphical representation on the interface means of the controller.

In another embodiment of the invention the graphical representation of the first pressure/volume relationship displayed on the interface means of the controller is displayed in the form of a graph, with the cavity pressure plotted on the ordinate or the abscissa, and the cavity volume being plotted on the other one of the ordinate and the abscissa, and the interface means is adapted to permit the desired working pressure value or a desired working cavity volume of the cavity to be inputted to the controller through the interface means for transmission to the insufflator by touching the graph at a point thereon corresponding to the pressure value or the cavity volume corresponding to the desired working pressure value or the desired working cavity volume or by positioning a cursor on the graph at the pressure value or cavity volume corresponding to the desired working pressure value or the desired working cavity volume.

In one embodiment of the invention a look-up table is displayed on the interface means of the controller, the look-up table displaying the first transition pressure value and a first cavity volume of the cavity of the subject corresponding to the first transition pressure value thereof and cross-referenced with the first transition pressure value, and the second transition pressure value and a second cavity volume of the cavity of the subject corresponding to the second transition pressure value thereof and cross-referenced with the second transition pressure value, and a plurality of intervening pressures of values lying between the first transition pressure value and the second transition pressure value and respective intervening cavity volumes of the cavity of the subject corresponding to the respective intervening pressure values thereof and cross-referenced with the corresponding respective intervening pressure values, and the interface means of the controller is adapted to permit entry therethrough to the controller of one of the pressure values or one of the cavity volumes displayed in the look-up table as the desired working pressure value or the desired working cavity volume for transmission to the insufflator.

Preferably, the interface means of the controller is adapted for entering the one of the pressure values or the one of the cavity volumes displayed in the look-up table thereof as the desired working pressure value or the desired working cavity volume to the controller by touching the pressure value or the cavity volume in the look-up table displayed in the interface means corresponding to the desired working pressure value to be selected or the desired working cavity volume to be selected, or by identifying the pressure value or the cavity volume in the look-up table displayed in the interface means by a cursor corresponding to the desired working pressure value to be selected or the desired working cavity volume to be selected.

In one embodiment of the invention the insufflator is operable under the control of the controller in a set-up mode, and the insufflator or the controller is operable to determine the first transition pressure value and the second transition pressure value as the insufflator is operating in the set-up mode.

In another embodiment of the invention the insufflator or the controller is programmed to determine the pressure/volume relationship between the cavity pressure and the volume of the cavity of the subject when the insufflator is operating in the set-up mode for determining the first transition pressure value and the second transition pressure value.

Preferably, the interface means of the controller is adapted for inputting a set-up mode request, and the controller is adapted to produce a set-up mode request signal for transmission to the insufflator to operate the insufflator in the set-up mode in response to inputting of the set-up mode request.

In another embodiment of the invention the interface means of the controller is adapted for inputting a normal insufflating mode request, and the controller is adapted to produce a normal insufflating mode request signal in response to the normal insufflating mode request for transmission to the insufflator to operate the insufflator in a normal insufflating mode for insufflating the cavity of the subject at the selected desired working pressure value.

In a further embodiment of the invention the insufflator is operable in the normal insufflating mode under the control of the controller.

In one embodiment of the invention the interface means of the controller is adapted for inputting a minimum safe working pressure value below which the cavity pressure should not fall during insufflating thereof, and the controller is adapted to transmit a signal to the insufflator indicative of the minimum safe working pressure value in response to the inputting of the minimum safe working pressure value, and the insufflator is programmed to store the minimum safe working pressure value and to maintain the cavity pressure above the minimum safe working pressure value during insufflating of the cavity, and preferably, the minimum safe working pressure value is at least not less than the first transition pressure value, and advantageously, the minimum safe working pressure value is greater than the first transition pressure value.

In one embodiment of the invention the minimum safe working pressure value is stored in memory in the controller, and preferably, the minimum safe working pressure value is stored in memory in the insufflator.

Preferably, the minimum safe working pressure value is determined by the surgeon at the second location, and advantageously, is entered into the controller by the surgeon at the second location through the interface means.

In another embodiment of the invention the insufflator or the controller is adapted for determining the minimum safe working pressure value.

In one embodiment of the invention the insufflator is adapted to produce a signal indicative of the cavity pressure falling to the minimum safe working pressure value for reception by the controller in response to the cavity pressure falling to the minimum safe working pressure value, and the controller is adapted to produce a human sensory perceptible signal indicative of the cavity pressure falling to the minimum safe working pressure value in response to the signal received from the insufflator indicative of the cavity pressure falling to the minimum safe operating value alerting to the cavity pressure having fallen to the minimum safe working pressure value.

In one embodiment of the invention the human sensory perceptible signal indicative of the cavity pressure falling to the minimum safe working pressure value produced by the controller comprises a visual signal, and in another embodiment of the invention the human sensory perceptible signal indicative of the cavity pressure falling to the minimum safe working pressure value produced by the controller comprises an audible signal.

In another embodiment of the invention the interface means of the controller is adapted for inputting a maximum safe working pressure value above which the cavity pressure should not exceed during insufflating thereof, and the controller is adapted to transmit a signal to the insufflator indicative of the maximum safe working pressure value in response to the inputting of the maximum safe working pressure value, and the insufflator is programmed to store the maximum safe working pressure value and to maintain the cavity pressure below the maximum safe working pressure value during insufflating of the cavity.

In one embodiment of the invention the maximum safe working pressure value is at least a pressure value just less than the value of the second transition pressure value, and preferably, the maximum safe working pressure value is approximately equal to the second transition pressure value, and in some embodiments of the invention the maximum safe working pressure value is a pressure value just greater than the second transition pressure value.

In one embodiment of the invention the maximum safe working pressure value is stored in memory in the controller, and preferably, the maximum safe working pressure value is stored in memory in the insufflator.

In another embodiment of the invention the maximum safe working pressure value is determined by the surgeon at the second location, and preferably, the maximum safe working pressure value is entered into the controller by the surgeon through the interface means of the controller.

In another embodiment of the invention the insufflator or the controller is adapted for determining the maximum safe working pressure value.

In another embodiment of the invention the insufflator is adapted to transmit a signal indicative of the cavity pressure reaching the maximum safe working pressure value for reception by the controller in response to the cavity pressure reaching the maximum safe working pressure value, and preferably, the controller is adapted to produce a human sensory perceptible signal indicative of the cavity pressure reaching the maximum safe working pressure value in response to the signal received from the insufflator indicative of the cavity pressure reaching the maximum safe working pressure value alerting to the cavity pressure having reached the maximum safe working pressure value.

In one embodiment of the invention the human sensory perceptible signal indicative of the cavity pressure having reached the maximum safe working pressure value produced by the controller comprises a visual signal, and in another embodiment of the invention the human sensor perceptible signal indicative of the cavity pressure having reached the maximum safe working pressure value comprises an audible signal.

In one embodiment of the invention the first and second communicating means are adapted for two-way communications between the controller and the insufflator.

The invention also provides an insufflating system for remotely insufflating a cavity in the body of a human or animal subject, the insufflating system comprising:
an insufflator located in a first location and connected to the cavity of the human or animal subject, the insufflator comprising a first communicating means,
a controller located in a second location remote from the first location, the controller comprising a second communicating means adapted for communicating with the first communicating means of the insufflator,
the controller being adapted to control the operation of the insufflator through the first and second communicating means for insufflating the cavity of the subject at a desired working pressure value at which the cavity is to be maintained, the desired working pressure value being a pressure value selectable from a range of pressure values between a first transition pressure value and a second transition pressure value,
the first transition pressure value being a pressure value in a pressure/volume relationship between the pressure in the cavity (cavity pressure) and the volume of the cavity of the subject at which the pressure/volume relationship transitions from an initial pressure/volume relationship during which the cavity pressure remains substantially constant while insufflating gas is being delivered to the cavity to a first pressure/volume relationship during which the cavity pressure increases per unit volume of insufflating gas delivered to the cavity, and
the second transition pressure value being a pressure value in the pressure/volume relationship at which the first pressure/volume relationship transitions to a second pressure/volume relationship during which the cavity pressure increases per unit volume of insufflating gas delivered to the cavity at a greater rate than the cavity pressure increases per unit volume of insufflating gas delivered to the cavity during the first pressure/volume relationship.

The advantages of the invention are many. By knowing the minimum working pressure value and the optimum maximum pressure value of the working pressure range for a cavity of a subject, a surgeon or clinician can readily identify the most appropriate working pressure value at which the cavity should be insufflated without any further trial or error. Once a surgeon or clinician knows the working pressure range within which the cavity of a subject should be insufflated, there is no danger of the cavity being over-insufflated or under-insufflated. Additionally, if during the carrying out of a procedure, the surgeon or clinician finds that the working volume within the cavity is more than adequate for the procedure being carried out, the cavity pressure may be reduced to a lower cavity pressure which would still provide adequate working volume in the cavity. Since the surgeon or clinician knows the working pressure range, when reducing the cavity pressure, there will be no danger of the surgeon or clinician reducing the cavity pressure below the minimum working pressure value.

Likewise, if during the carrying out of a procedure the surgeon or clinician believes an increase in the working volume within the cavity is required, a surgeon or clinician will readily know whether a further increase in the cavity pressure will produce a corresponding increase in the working volume of the cavity. In other words, if the cavity pressure is below the optimum maximum pressure value, then an increase in the cavity pressure will produce an increase in the working volume within the cavity. However, if the cavity pressure is already at the optimum maximum pressure value, an increase in the cavity pressure would yield minimal if any increase in the working volume of the cavity.

A further advantage of the invention is provided when a working volume range for the cavity is provided, in that a surgeon or clinician may select a working volume value instead of a working pressure value at which the cavity is to be insufflated.

The advantages of the insufflating system and method according to the invention for remotely insufflating a cavity in the body of a human or an animal subject are many. A particularly important advantage of the insufflating system and method is that it allows a surgeon to control insufflating of a cavity in the body of a human or an animal subject in which a minimally invasive procedure is being carried out at a location remote from the location at which the subject is located, thereby enabling the surgeon to accurately control the insufflating of the cavity of the subject, such that the cavity is maintained insufflated at an optimal safe working pressure consistent with adequate visualisation in the cavity. By providing a surgeon at the second location remote from the first location at which the subject is located with either the first transition pressure value and the second transition pressure value of the range of pressure values of the first pressure/volume relationship between the cavity pressure and the cavity volume of the subject, or a graphical representation of the first pressure/volume relationship between the cavity pressure and the cavity volume of the subject, the surgeon can readily determine the most appropriate pressure to which the cavity should be insufflated consistent with adequate visualisation in the cavity.

Furthermore, with the knowledge of the first and second transition pressure values and/or a graphical representation of the first pressure/volume relationship, a surgeon carrying out a procedure remotely of the location at which the subject is located may readily easily determine the maximum safe working pressure value above which the cavity pressure should not exceed and the minimum safe working pressure value below which the cavity pressure should not fall during insufflating of the cavity. The maximum and minimum safe working pressure values may then be entered into the insufflator through the controller by the surgeon at the remote location, so that in the event of the cavity pressure exceeding the maximum safe working pressure value or falling below the minimum safe working pressure value, a surgeon at the remote location can be alerted by an appropriate alert signal transmitted from the insufflator to the controller which may be produced by the controller as a visual warning signal or an audible warning signal.

Alerting the surgeon at the remote location to the cavity pressure exceeding the maximum safe working pressure value or falling below the minimum safe working pressure value is particularly advantageous. Alerting the surgeon at the remote location to the cavity pressure falling below the minimum safe working pressure value has a particularly important advantage, due to the fact that during the carrying out of a minimally invasive procedure in a cavity, and in particular minimally invasive surgical procedures, a number of tools may be located in the cavity, and if the cavity pressure were to fall below the minimum safe working pressure value resulting in the collapse of the cavity, serious injury to the cavity and to the subject could arise if the cavity collapsed onto a tool or tools therein. Thus, by being alerted to the cavity pressure either exceeding the maximum safe working pressure value or falling below the minimum safe working pressure value, a surgeon at a remote location can take corrective action by inputting an appropriate signal into the controller through the interface means to appropriately operate the insufflator to decrease or increase the cavity pressure as appropriate.

Needless to say, apart from those particularly important advantages of the insufflating system and method for remotely insufflating a cavity in the body of a subject according to the invention, the insufflating system comprises many if not all the advantages already described with reference to the insufflators described, particularly, the advantage of being able to accurately select a working pressure value up to but preferably slightly below the optimum maximum pressure value or the maximum safe working pressure value to which the peritoneal cavity of a subject may be insufflated, and to also enable a surgeon to accurately determine the minimum safe working pressure value and the maximum safe working pressure value for a cavity, and in particular, for a peritoneal cavity of a subject.

The invention will be more clearly understood from the following description of some preferred embodiments thereof, which are given by way of example only, with reference to the accompanying drawings, in which:

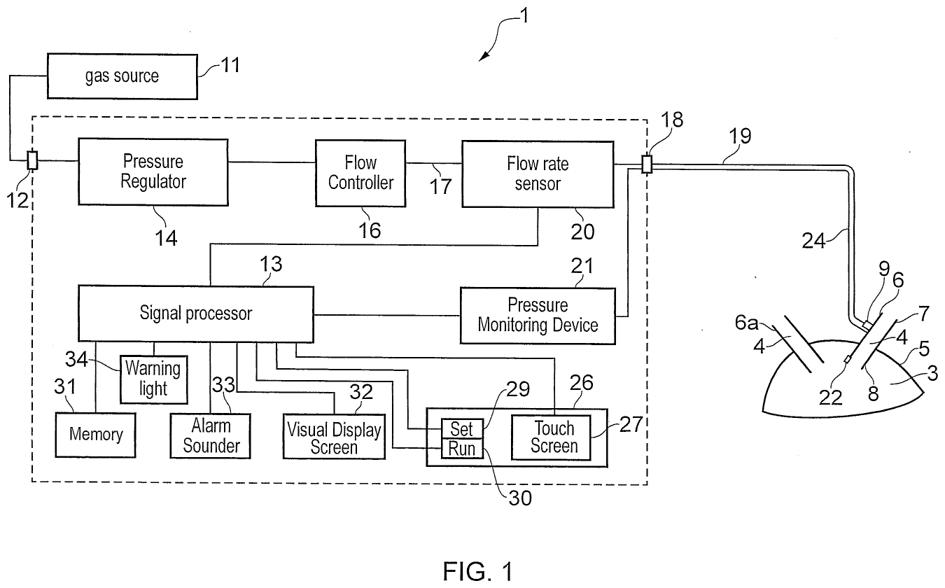

FIG. 1 is a block representation of an insufflator according to the invention,

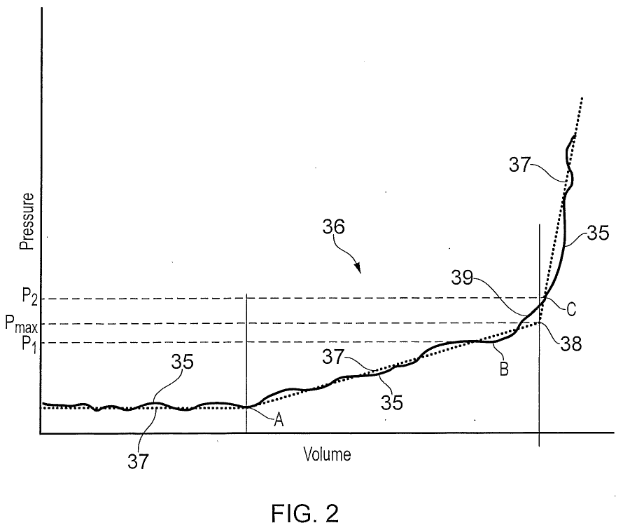

FIG. 2 is graphical representation of a pressure/volume relationship between pressure in the cavity in the body of a subject being insufflated by the insufflator of FIG. 1 and the cumulative volume of insufflating gas delivered to the cavity with volume plotted on the abscissa and pressure plotted on the ordinate,

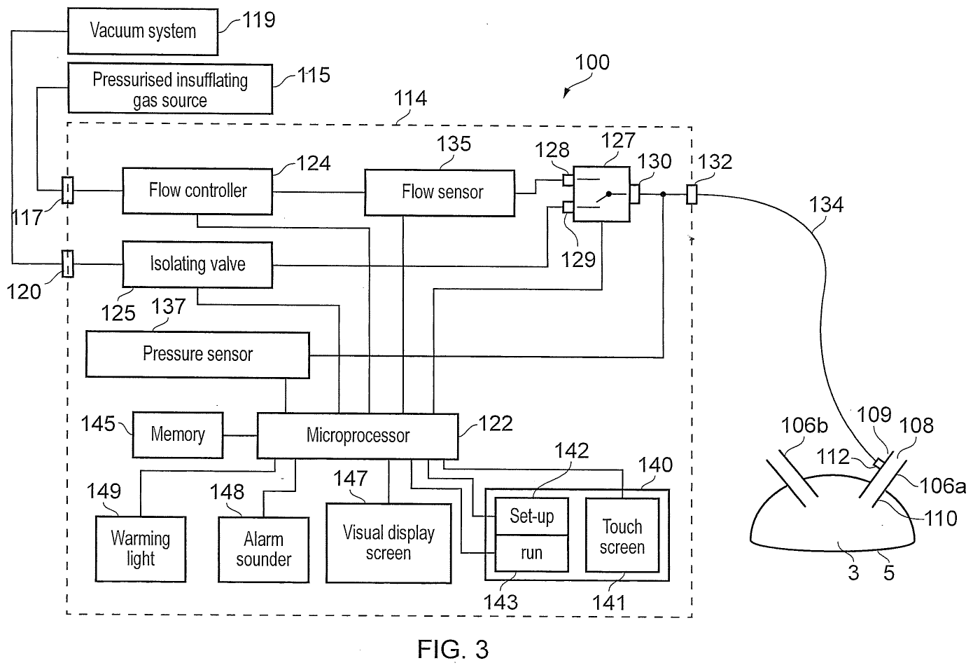

FIG. 3 is a block representation of an insufflator according to another embodiment of the invention,

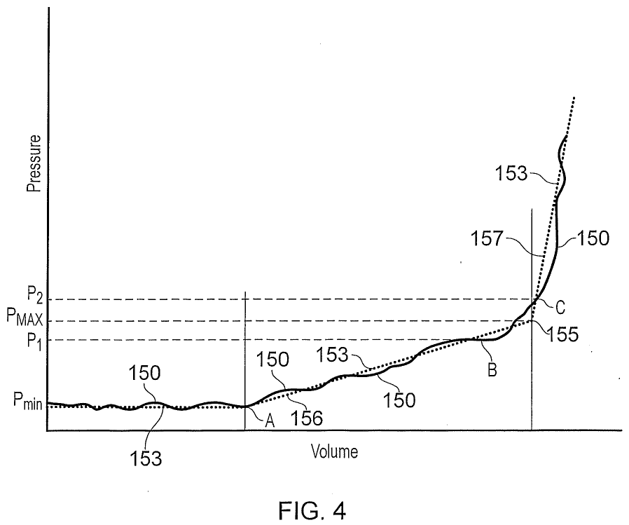

FIG. 4 is a graphical representation of a pressure/volume relationship between pressure in the cavity in the body of a human or animal subject being insufflated by the insufflator of FIG. 3 and the cumulative volume of insufflating gas delivered to the cavity with the volume plotted on the abscissa and pressure plotted on the ordinate,

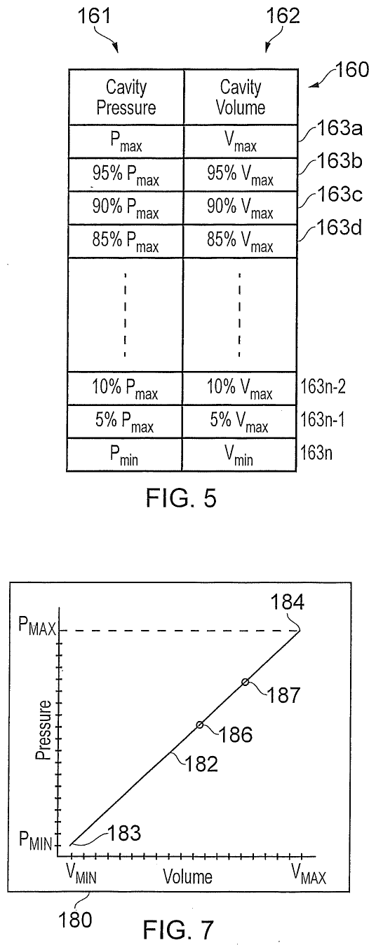

FIG. 5 is a look-up table of the insufflator of FIG. 3 prepared from the pressure/volume relationship of FIG. 4,

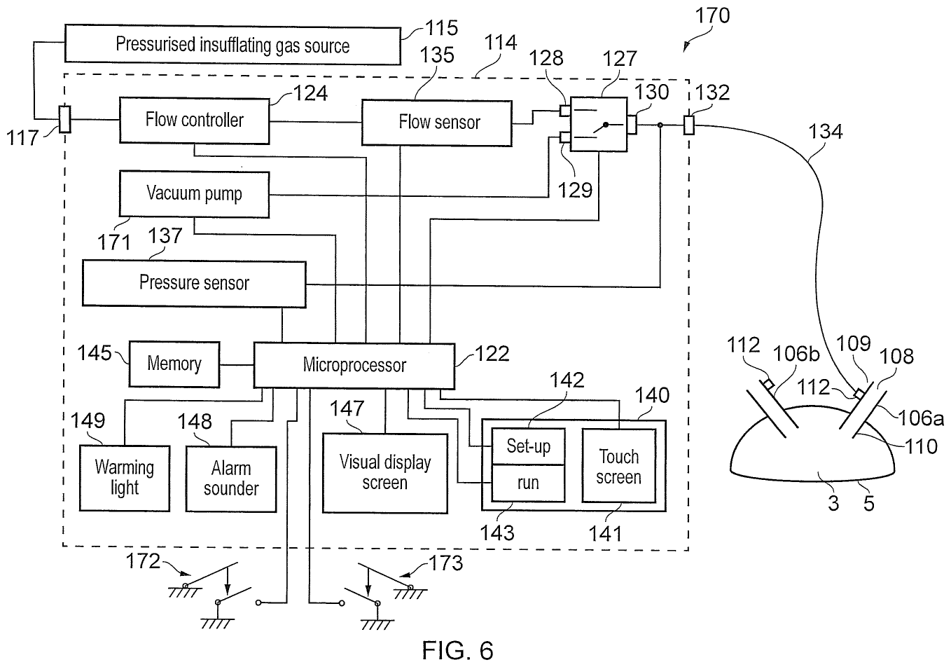

FIG. 6 is a block representation of an insufflator according to another embodiment of the invention,

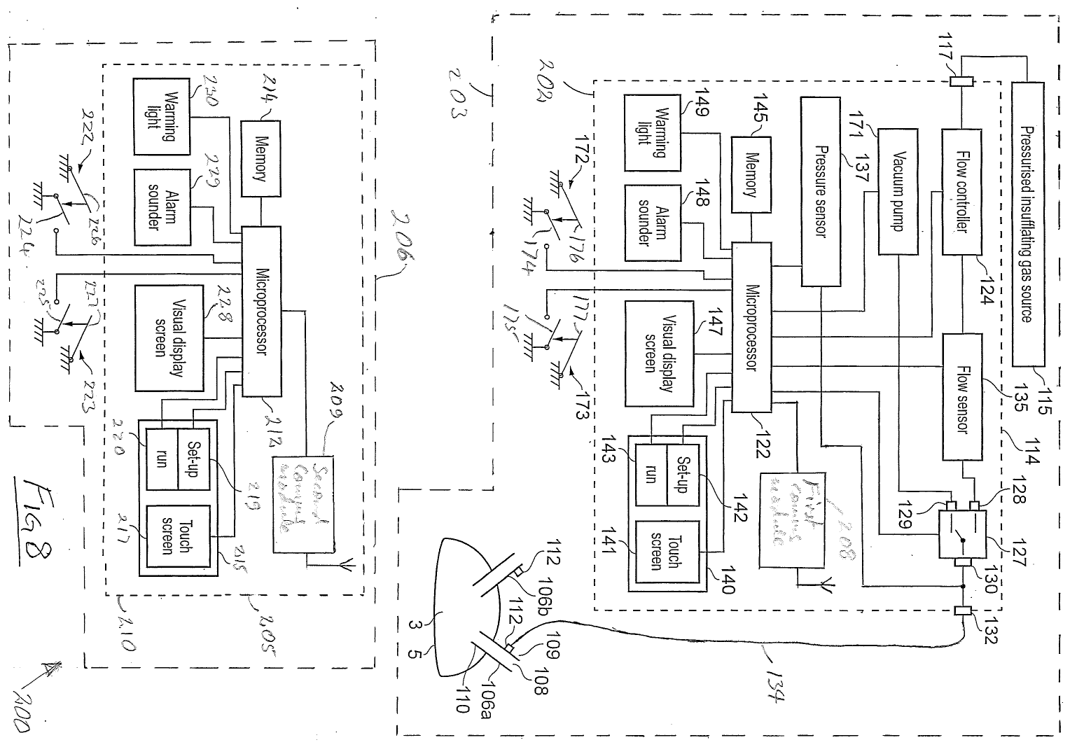

FIG. 7 is an illustration of an interface of an insufflator according to another embodiment of the invention, and

FIG. 8 is a block representation of an insufflating system also according to the invention for remotely insufflating a peritoneal cavity in the body of a human or animal subject.

Referring to the drawings and initially to FIGS. 1 and 2 thereof, there is illustrated an insufflator according to the invention indicated generally by the reference numeral 1 for insufflating a cavity, in this case the peritoneal cavity 3 in the body 5 of a human or animal subject during a minimally invasive surgical or investigative procedure being carried out, typically, laparoscopically. Although, it will be readily apparent to those skilled in the art that the insufflator 1 may be used for insufflating any lumen, vessel or cavity in the body of a human or animal subject, laparoscopically, endoscopically or otherwise. The insufflator 1 as will be described in detail below is operable in two modes, namely, in a normal insufflating mode and in a set-up mode. In the normal insufflating mode the insufflator 1 delivers insufflating gas to the cavity 3 through a trocar 6 for insufflating the cavity 3 and for maintaining the cavity insufflated during the surgical or investigative procedure. In the set-up mode of the insufflator 1 an optimum maximum pressure value, above which the cavity 3 of the subject should ideally not be insufflated, is determined prior to commencement of operating of the insufflator 1 in the normal insufflating mode for insufflating the cavity 3 of that subject. The optimum maximum pressure value is a pressure at which any further delivery of insufflating gas into the cavity of the subject would result in a further increase of the pressure in the cavity with little or no further increase in the working volume in the cavity.

The trocar 6 comprises an instrument channel 4 extending therethrough, and extends from a proximal end 7 to a distal end 8, which is located in the cavity 3, and may or may not comprise an insufflating gas inlet port 9 to which insufflating gas is delivered to the trocar 6. A bore (not shown) extending along the wall of the trocar 6 from the inlet port 9 to the distal end 8 of the trocar 6 accommodates insufflating gas from the inlet port 9 into the cavity 3. Alternatively, the insufflating gas may be delivered through a Veress needle.

The insufflator 1 comprises a housing 10, which may include a source of insufflating gas, which if included in the housing 10 would normally be provided in a pressurised container containing pressurised insufflating gas, typically, carbon dioxide. However, in this embodiment of the invention the insufflator 1 is adapted to receive the pressurised insufflating gas from an external source 11, which typically, comprises compressed carbon dioxide, typically, from a source of compressed carbon dioxide available in a hospital operating theatre, or elsewhere where the insufflator 1 is being operated. An inlet port 12 is provided in the housing 10 for connecting the insufflator 1 to the external pressurised source 11 of the insufflating gas. A signal processor, in this case provided by a microprocessor 13 located in the housing 10 controls the operation of the insufflator 1 as will be described below. However, it will be appreciated that any other suitable form of signal processor may be provided, for example, a microcontroller or other such suitable signal processor.

A pressure regulator 14 located in the housing 10 is connected to the inlet port 12 for receiving the insufflating gas therefrom, and for stepping down the pressure of the insufflating gas to a pressure not exceeding 40 mmHg.

A delivery means comprising a flow controller 16 located in the housing 10, controls the flow rate of the insufflating gas to the cavity 3 of the subject, and in turn the pressure in the cavity 3. The flow controller 16 is connected to the pressure regulator 14 and receives the insufflating gas from the pressure regulator 14 at the stepped down pressure. The flow controller 16 is operated under the control of the microprocessor 13 for controlling the flow rate at which the insufflating gas is delivered to the cavity 3 of the subject as will be described below.

A connecting tube 17 connects the flow controller 16 to an outlet port 18 of the housing 10. A gas line 19 from the outlet port 18 supplies the insufflating gas to the cavity 3 of the subject through the trocar 6. The gas line 19 may be entered directly into the cavity 3 through the instrument channel 4 in the trocar 6, or if the trocar 6 is provided with the gas inlet port 9, the gas line 19 may be connected to the gas inlet port 9, through which the insufflating gas is delivered into the cavity 3.

A flow rate sensor 20 located in the housing 10 in the connecting tube 17 monitors the flow rate of the insufflating gas through the connecting tube 17 to the cavity 3, and produces a signal indicative of the rate at which the insufflating gas is being delivered to the cavity 3. The microprocessor 13 is programmed to read the value of the signal produced by the flow rate sensor 20 and to compute the cumulative volume of the insufflating gas delivered to the cavity 3 from the commencement of delivery of insufflating gas thereto.

A pressure monitoring device 21 located in the housing 10 monitors cavity pressure, namely, the pressure in the cavity 3 of the subject and produces a signal indicative of the pressure in the cavity 3. The pressure monitoring device 21 reads signals from a pressure sensor 22 which may be located in the housing 10 in the connecting tube 17 downstream of the flow sensor 20, or may be located in the cavity 3, for example, on a portion of the trocar 6 located within the cavity 3, which would give a direct reading of the cavity pressure. If the pressure sensor were located in the connecting tube 17 downstream of the flow sensor 20, during monitoring of the cavity pressure, in order to determine the cavity pressure, either the flow controller 16 would isolate the cavity 3 from the insufflating gas to give a true value of the pressure in the cavity 3, or alternatively, the pressure of the insufflating gas flowing through the connecting tube 17 would be read from the pressure sensor. The pressure monitoring device 21 would then apply a compensating factor to the pressure value read from the pressure sensor to obtain the true value of the cavity pressure. However, in order to determine the value of the compensating value to be applied to the signals indicative of the read pressure value of the insufflating gas flowing in the connecting tube 17, the pressure monitoring device would obtain the flow rate of the insufflating gas in the connecting tube 17 from either the flow sensor or the microprocessor 13, and would apply a suitable compensating value to the signal read from the pressure sensor taking account of the read pressure value, the flow rate of the insufflating gas in the connecting tube 17, as well as the frictional resistance to flow in the connecting tube 17 and the gas line 19 between the pressure sensor and the trocar 6.

Alternatively, the pressure sensor, if located in the housing 10 may be connected directly to the cavity 3 through a pressure monitoring conduit (not shown) which would extend from the pressure sensor through the trocar 6 into the cavity 3, or through a second trocar 6a, similar to the trocar 6 extending into the cavity 3, so that the pressure monitored by the pressure sensor would be the true pressure in the cavity 3.

In this embodiment of the invention the pressure sensor 22 is located on an outer surface of the trocar 6 adjacent the distal end 8 thereof, and produces a signal indicative of the cavity pressure. An electrically conductive wire 24 connects the pressure sensor 22 to the pressure monitoring device 21 to apply the signal from the pressure sensor 22 to the pressure monitoring device 21. The wire 24 extends along and is secured to the outer surface of the gas line 19 and enters the housing 10 with the gas line 19 at the outlet port 18, and then extends to and is connected to the pressure monitoring device 21. However, it is envisaged that in some embodiments of the invention the communication between the pressure sensor 22 and the pressure monitoring device 21 may be provided wirelessly.

The microprocessor 13 is programmed to control the flow controller 16 to control the rate of flow of insufflating gas to the cavity 3 to maintain the pressure in the cavity 3 at a selected working pressure, typically in the range of 10 mmHg to 15 mmHg, when the microprocessor 13 is controlling the insufflator 1 to operate in the normal insufflating mode.

An interface means, namely, an interface 26, which may comprise a touch screen, a keypad or any other suitable interface, and which in this case comprises a touch screen 27, is located on the housing 10 and acts as a main input means to enable entry of data into the microprocessor 13, such as, the value of the selected working pressure value, at which the cavity pressure is to be maintained during insufflating of the cavity 3 when the insufflator 1 is operating in the normal insufflating mode, and other relevant data, for example, the maximum safe pressure value to which the cavity 3 may be insufflated in the normal insufflating mode, and the maximum set-up safe pressure value when the insufflator 1 is operating in the set-up mode. The maximum safe pressure value in the normal insufflating mode and the maximum set-up safe pressure value may be the same or different, and depending on the cavity being insufflated may range between 25 mmHg and 30 mmHg or between 10 mmHg and 15 mmHg. In some embodiments of the invention the maximum safe pressure value may range between 20 mmHg and 25 mmHg. Although, in some embodiments of the invention the maximum safe pressure value in the normal insufflating mode and the maximum set-up safe pressure value may be pre-set in the microprocessor 15. The touch screen 27 also displays data relevant to the operation of the insufflator 1, such as the optimum maximum pressure value and other data relevant to the operation of the insufflator 1.

The interface 26 also comprises a pair of button operated switches, namely, a set-up mode select button operated switch 29 and a normal insufflating run mode select button operated switch 30 for selecting the operational modes of the insufflator 1, namely, the set-up mode or the normal insufflating mode, respectively. The microprocessor 13 reads signals from the touch screen 27 and from the set-up mode and normal insufflating run mode select button operated switches 29 and 30 of the interface 26. It is envisaged that instead of the interface comprising a touch screen and a separate set-up mode select button operated switch and a separate normal insufflating run mode select button operated switch, a graphic user touch screen interface may be provided, which would comprise a graphic display of representations of such set-up and normal insufflating run mode select button operated switches operable by touching the corresponding graphic representation thereof.

A memory 31 of the microprocessor 13 which may be any suitable electronic memory such as a random access memory is provided for storing data for access by the microprocessor 13 as will be described below. The microprocessor 13 controls a visual display screen 32 which provides visual data to a surgeon and clinician personnel in an operating theatre. An alerting device for producing an alerting signal is also operated under the control of the microprocessor 13 in the event of the cavity pressure exceeding either of the maximum safe pressures, or as will be described below, the optimum maximum pressure value. The alerting device, in this case comprises both an alarm sounder 33 and a warning light 34 mounted on the housing 10 for producing an audible alerting signal and a visual alerting signal, respectively.

Turning now to the operation of the insufflator 1, the insufflator 1 is connected to the external pressurised source 11 of insufflating gas through the inlet port 12. The gas line 19 is connected to the gas inlet port 9 of the trocar 6 which has already been inserted into the cavity 3 of the subject for supplying insufflating gas to the cavity through the trocar 6. Alternatively, the gas line 19 may be entered directly into the cavity 3 through the trocar 6. The wire 24 from the pressure sensor 22 is connected to the pressure monitoring device 21 through the outlet port 18 of the housing 10.

Initially, the insufflator 1 is operated in the set-up mode to determine the optimum maximum pressure, above which the cavity 3 of the subject should ideally not be inflated. The set-up select button operated switch 29 is operated to set the microprocessor 13 to operate in the set-up mode, and in turn to operate the insufflator 1 in the set-up mode. In the set-up mode, the microprocessor 13 controls the flow controller 16 to deliver insufflating gas to the cavity 3 of the subject at a constant rate, typically in the range of 0.5 litres per minute to 5 litres per minute, and reads the values of the signal from the flow sensor 20 and the values of the signal from the pressure monitoring device 21 at predefined time intervals, typically of 10 milliseconds as the insufflating gas is being slowly delivered to the cavity 3.

As the values of the signals are read from the flow sensor 20 and the pressure monitoring device 21 at the end of each predefined time interval, the microprocessor 13 is programmed to determine the cumulative volume of insufflating gas delivered to the cavity 3 from the commencement of delivery of the insufflating gas to the cavity 3 and the corresponding pressure in the cavity 3. The determined cumulative volume of insufflating gas delivered to the cavity 3 and the corresponding pressure in the cavity 3 are then time-stamped, cross-referenced and stored in the memory 31 of the microprocessor 13. The microprocessor 13 is programmed to compute a pressure/volume relationship between the pressure in the cavity 3 and the cumulative volume of insufflating gas delivered to the cavity 3 at the end of each predefined time interval, as each new value of the cumulative volume of the insufflating gas delivered to the cavity 3 and the corresponding value of pressure in the cavity 3 is determined, and each computed pressure/volume relationship is time-stamped and stored in the memory 31. Additionally, at the end of each predefined time interval, the microprocessor 13 is programmed to compute a value of the increase in pressure in the cavity 3 per unit volume of insufflating gas delivered to the cavity 3, and each computed value of the increase in the pressure in the cavity 3 per unit volume of insufflating gas delivered to the cavity 3 and the corresponding value of the pressure in the cavity are time-stamped, cross-referenced and stored in memory 31. The microprocessor 13 is programmed when computing the value of the increase in the pressure in the cavity per unit volume of insufflating gas delivered to the cavity to apply a smoothing algorithm to the computation to smooth the computed values. The microprocessor 13 is also programmed when computing each value of the pressure/volume relationship to apply a smoothing algorithm to the computation. In this embodiment of the invention the smoothing algorithms comprise respective moving average algorithms, although any other smoothing algorithms may be used.

Before describing the steps carried out by the microprocessor 13 in the set-up mode further to determine the optimum maximum pressure value, reference is now made to FIG. 2 in which a line 35 of a graph 36 represents a graphical relationship of the actual pressure/volume relationship between the cumulative volume of insufflating gas delivered to the cavity 3 and the pressure in the cavity 3. The pressure in the cavity 3 is plotted on the ordinate, and the cumulative volume of the insufflating gas delivered to the cavity 3 is plotted on the abscissa of the graph 36. A line 37 representing a smoothed version of the pressure/volume relationship of the line 35, after the moving average algorithm has been applied to the pressure/volume relationship of the line 35 is also illustrated in FIG. 2.

As can be seen from the lines 35 and 37 of the pressure/volume relationship between the cavity pressure and the cavity volume, during an initial pressure/volume relationship from the commencement of delivery of insufflating gas to the cavity 3 to the point A on the line 35 of the pressure/volume relationship, as insufflating gas is being delivered to the cavity 3, the pressure of the cavity 3 remains substantially constant as the cumulative volume of the insufflating gas which is delivered to the cavity 3 increases, until the cavity 3 has been filled with the insufflating gas at the point A on the line 35. At this point, namely, at the point A on the line 35, as further insufflating gas is delivered to the cavity 3, the cavity pressure commences to rise, thereby establishing a first pressure/volume relationship between the cavity pressure and the cumulative volume of insufflating gas delivered to the cavity 3. In other words, the pressure/volume relationship between the cavity pressure and the cavity volume transitions from the initial pressure/volume relationship to the first pressure/volume relationship at the point A on the line 35 of FIG. 2. The first pressure/volume relationship continues from the point A to the point B, during which the first pressure/volume relationship is a substantially linear relationship with the pressure in the cavity 3 increasing as the cumulative volume of insufflating gas delivered to the cavity 3 increases, and with the value of the increase in pressure in the cavity 3 per unit volume of insufflating gas delivered to the cavity 3 being of a first substantially constant value, which is greater than zero. The slope of the line 35 represents the increase in pressure in the cavity 3 per unit volume of insufflating gas delivered to the cavity 3.

At point B on the line 35, the substantially linear first pressure/volume relationship transitions to an intermediate pressure/volume relationship, which is non-linear, and during which the value increase in the pressure in the cavity 3 per unit volume of insufflating gas delivered to the cavity 3 progressively increases. The non-linear intermediate pressure/volume relationship continues to point C on the line 35. At point C of the line 35, the non-linear intermediate pressure/volume relationship transitions to a second pressure/volume relationship comprising a substantially linear relationship with the pressure in the cavity 3 increasing as the cumulative volume of insufflating gas in the cavity 3 increases, and during which the value of the increase in the pressure in the cavity 3 per unit volume of insufflating gas delivered to the cavity 3 is of a second substantially constant value, which is significantly greater than the first substantially constant value of the increase in pressure in the cavity 3 per unit volume of insufflating gas delivered to the cavity 3 of the first pressure/volume relationship.

Thus, from point B on the line 35 at the end of the first pressure/volume relationship, the pressure in the cavity 3 commences to rise from a first pressure value P₁ at a non-linear ever-increasing rate per unit volume of gas delivered to the cavity 3 of the subject to a second pressure value P₂ at the point C on the line 35 at the commencement of the second pressure/volume relationship. Accordingly, the gain in working volume in the cavity 3 per unit volume of gas delivered to the cavity begins to decrease from the point B, while the increase in pressure per unit volume of insufflating gas delivered to the cavity 3 begins to increase. Thus, at some point between the first pressure value P₁ at point B of the line 35 and the second pressure value P₂ at point C of the line 35, and onward from the point C, the gain in working volume in the cavity 3 per unit volume of gas delivered to the cavity 3 is marginal while the pressure in the cavity 3 per unit volume of insufflating gas delivered to the cavity commences to rapidly increase.

The microprocessor 13 is programmed to determine the optimum maximum pressure at which the gain in working volume in the cavity 3 per unit volume of insufflating gas delivered to the cavity 3 with respect to the pressure in the cavity is optimised. In this embodiment of the invention the microprocessor 13 is programmed to determine the optimum maximum pressure value as the pressure at the point of intersection 38 of the portion of the smoothed line 37 representative of the first pressure/volume relationship and the portion of the smoothed line 37 representative of the second pressure/volume relationship. The microprocessor 13 is programmed to determine the point of intersection 38 of the portions of the smoothed line 37 representative of the first pressure/volume relationship and the second pressure/volume relationship by either interpolating between the two portions representative of the first and second pressure/volume relationships, or by extrapolating the portion of the smoothed line 37 representative of the first pressure/volume relationship forward beyond the value of the pressure P₁, and by extrapolating the portion of the smoothed line 37 representative of the second pressure/volume relationship backwards from the value of the pressure P₂.

The point of intersection 38 on the line 37 approximates to the point of inflection of the line 35, at which the first pressure/volume relationship transitions to the second pressure/volume relationship, and the value of the pressure in the cavity 3 at the point of intersection 38 on the line 37 is the value of the transition pressure at which the first pressure/volume relationship transitions to the second pressure/volume relationship. The microprocessor 13 is programmed to determine the value of the pressure in the cavity 3 at the point of intersection 38 as the transition pressure value, and to store the value of the transition pressure in the memory 31 as the value of the optimum maximum pressure Pₘₐₓ for the cavity 3, above which the insufflator 1 should ideally not insufflate the cavity 3, since there is little or no gain in the working volume in the cavity 3 once the pressure in the cavity 3 reaches the value of the optimum maximum pressure Pₘₐₓ. The microprocessor 13 is programmed to output a signal indicative of the value of the optimum maximum pressure Pₘₐₓ to the visual display screen, and the value of the optimum maximum pressure Pₘₐₓ is displayed on the visual display screen 32 under the control of the microprocessor 13. Additionally, the microprocessor 13 may be programmed, when running in the normal insufflating mode, to control the flow controller 16 to prevent the pressure in the cavity 3 exceeding the optimum maximum pressure value, as will be described below.

If at any stage, while the insufflator 1 is operating in the set-up mode, the pressure in the cavity 3 exceeds the maximum set-up safe pressure value, the microprocessor 13 is programmed to operate the flow controller 16 to terminate delivery of insufflating gas to the cavity 3.

On the value of the optimum maximum pressure value being determined, insufflating of the cavity 3 may be terminated, or insufflating of the cavity 3 may be continued, if the insufflator 1 is to be immediately switched to operate in the normal insufflating mode, as will be described below.

In an alternative embodiment of the invention the microprocessor 13 is programmed to determine the value of the optimum maximum pressure Pₘₐₓ, beyond which the cavity 3 should ideally not be insufflated, by determining the first pressure value P₁ at which the first pressure/volume relationship transitions to the intermediate pressure/volume relationship, and also by determining the second pressure value P₂ at which the intermediate pressure/volume relationship transitions to the second pressure/volume relationship, and then determining the average value of the first and second pressure values P₁ and P₂. In this embodiment of the invention the microprocessor 13 is programmed at the end of each predefined time interval to compute the value of the increase in the pressure in the cavity 3 per unit volume of insufflating gas delivered to the cavity 3, which is equivalent to the slope of the line 35 of the graph 36. However, in order to enable the first and second pressure values P₁ and P₂ to be determined more accurately, at the end of each predefined time interval, when computing the increase in the pressure in the cavity 3 per unit volume of insufflating gas delivered to the cavity 3, a smoothing algorithm, which in this case is also a moving average algorithm, is applied to the computation. Although, any suitable smoothing algorithm may be used. As each value of the increase in pressure in the cavity 3 per unit volume of insufflating gas delivered to the cavity is being computed, the corresponding value of the pressure in the cavity 3 is read and each corresponding pair of the values of the computed increase in pressure in the cavity per unit volume of insufflating gas delivered to the cavity and the pressure in the cavity are time-stamped, cross-referenced and stored in the memory 31.

As successive values of the increase in pressure in the cavity 3 per unit volume of insufflating gas delivered to the cavity 3 are computed, the signal processor is programmed to compare each computed value of the increase in the pressure in the cavity 3 per unit volume of insufflating gas delivered to the cavity 3 with the previously computed value or a number of the previously computed values of the increase in pressure in the cavity per unit volume of insufflating gas delivered to the cavity, to determine the first pressure value P₁. The microprocessor 13 is programmed to determine the first pressure value P₁ as the pressure in the cavity 3 when the computed values of the increase in pressure in the cavity 3 per unit volume of insufflating gas delivered to the cavity 3 ceases to be substantially constant. In other words, the first pressure value P1 is determined as the value of the pressure in the cavity 3 just prior to the value of the increase in pressure in the cavity per unit volume of insufflating gas delivered to the cavity beginning to increase.

Once the first pressure value P₁ has been determined, the microprocessor 13 is programmed to continue computing the values of the increase in the pressure in the cavity per unit volume of insufflating gas delivered to the cavity at the ends of the respective predefined time periods. On each value of the increase in pressure in the cavity 3 per unit volume of insufflating gas delivered to the cavity 3 being computed, the microprocessor 13 is programmed to compare the computed value with the previous or a number of the previously computed values of the increase in the pressure in the cavity per unit volume of insufflating gas delivered to the cavity. The microprocessor 13 is programmed to determine the second pressure value P₂ as being the pressure in the cavity 3 at which the first of the computed values of the increase in the pressure in the cavity 3 per unit volume of insufflating gas delivered to the cavity 3 becomes substantially constant again.

When the first and second pressure values P₁ and P₂ have been determined, the microprocessor 13 is programmed to determine the average value of the first and second pressure values P₁ and P₂, and the average value of the first and second pressure values P₁ and P₂ is determined as being the value of the optimum maximum pressure Pₘₐₓ beyond which the cavity 3 should ideally not be insufflated.

Once the value of the optimum maximum pressure Pₘₐₓ has been determined, the insufflator 1 is switched from the set-up mode to the normal insufflating mode by operating the normal insufflating run mode select button operated switch 30 to operate the insufflator 1, and in turn, the microprocessor 13, in the normal insufflating mode. If the desired value of the working pressure at which the cavity 3 is to be insufflated and the maximum safe pressure value to which the cavity 3 may be safely insufflated during operation of the insufflator 1 in the normal insufflating mode have not yet been entered, both the desired value of the working pressure and the value of the maximum safe pressure are entered in the microprocessor 13 through the touch screen 27. The desired working pressure value will, in general, be selected as a pressure value below the value of the optimum maximum pressure.

In the normal insufflating mode, the microprocessor 13 is programmed to control the operation of the flow controller 16 to supply insufflating gas to the cavity so that the pressure in the cavity 3 is maintained at the selected desired working pressure. The microprocessor 13 reads the value of the signal produced by the pressure monitoring device 21 and controls the flow controller 16 to deliver insufflating gas to the cavity at the appropriate flow rate in order to maintain the pressure in the cavity at the selected desired working pressure value.

If the surgeon inputs a signal through the touch screen 27 requesting an increase in the working pressure in the cavity, the microprocessor 13 controls the flow controller 16 to increase the flow rate of insufflating gas to the cavity 3 to increase the working pressure in the cavity 3. The microprocessor 13 controls the operation of the flow controller 16 to increase the flow rate of the insufflating gas to the cavity 3 in incremental steps and reads the value of the signal produced by the pressure monitoring device 21 to check that the pressure in the cavity 3 is raising as requested. The microprocessor 13 controls the flow controller 16 in response to the value of the signal read from the pressure monitoring device 21 until the working pressure has been raised to the working pressure selected by the surgeon or until the surgeon indicates that no further increase in the pressure in the cavity 3 is required.

During raising of the pressure in the cavity 3, at the predefined time intervals, the microprocessor 13 compares the value of the pressure in the cavity 3 read from the pressure monitoring device 21 with the stored value of the optimum maximum pressure. In the event of the pressure in the cavity 3 reaching the optimum maximum pressure, the microprocessor 13 outputs an alert signal to the sounder 33 and the warning light 34 to alert the surgeon to the fact that the pressure in the cavity 3 has reached the optimum maximum pressure. The microprocessor 13 also outputs the alert signal to the visual display screen 32 for displaying the pressure in the cavity 3 on the visual display screen 32, and also controls the visual display screen 32 to present a message indicating that the pressure in the cavity 3 has reached the optimum maximum pressure. The microprocessor 13 operates the flow controller 16 to terminate the supply of insufflating gas to the cavity 3 until the pressure in the cavity 3 has fallen back to the optimum maximum pressure.

Additionally, the microprocessor 13 may be programmed to allow a surgeon override the signal to the flow controller 16 terminating supply of insufflating gas to the cavity 3, and to allow the microprocessor 13 to operate the flow controller 16 to deliver insufflating gas to the cavity for further rising the pressure therein above the optimum maximum pressure value.

However, should the pressure in the cavity be raised to a level where the pressure in the cavity reaches the maximum safe pressure value, or the maximum safe set-up pressure value when the insufflator is operating in the set-up mode, the microprocessor 13 outputs a further alert signal to the sounder 33 and the warning light 34 to warn the surgeon that the pressure in the cavity 3 has reached the maximum safe pressure value, or the maximum safe set-up pressure value, as the case may be, and also outputs a signal to the visual display screen 32 for displaying the current pressure in the cavity 3 along with a warning message warning the surgeon that the pressure in the cavity 3 has reached either of the maximum safe working pressures. The microprocessor 13 may be programmed to operate the flow controller 16 to terminate delivery of insufflating gas to the cavity 3 until the cavity pressure has fallen below the relevant maximum safe pressure value.

In another embodiment of the invention the microprocessor may be programmed to allow insufflating of the cavity 3 beyond the maximum safe pressure, but only by direct intervention by the surgeon inputting an appropriate signal through the touch screen 27.

On completion of the procedure, insufflating of the cavity 3 is terminated, and the insufflating gas is evacuated from the cavity. The gas line 19 is disconnected from the trocar 6 or withdrawn through the trocar 6. The wire 24 is also disconnected from the trocar 6 and from the pressure sensor 22 mounted on the trocar 6.

Referring now to FIGS. 3 to 5 thereof, there is illustrated an insufflator according to another embodiment of the invention and indicated generally by the reference numeral 100 for insufflating a cavity, in this case, the peritoneal cavity 3 in the body 5 of a human or animal subject in this case a human subject during a minimally invasive surgical or investigative procedure being carried out, typically, laparoscopically. The cavity 3 and the body 5 of the subject are similar to the cavity 3 and the body 5 of the subject of FIG. 1. Although, it will be readily apparent to those skilled in the art that the insufflator 100 may be used for insufflating any other cavity, for example, a lumen, vessel, or hollow organ in the body of a human or animal subject laparoscopically, endoscopically or otherwise. In this embodiment of the invention the insufflator 100 as will be described below is configured so that the pressure or the volume to which the cavity 3 is to be insufflated are selectable over a range of working pressure values or working volume values. The insufflator 100 as will also be described in detail below is operable in two modes similar to the insufflator 1, namely, in a normal insufflating mode, in which the working pressure or the working volume to which the cavity 3 is to be insufflated are selectable in the working pressure range, or working volume range, and in a set-up mode in which a minimum working pressure of the working pressure range and an optimum maximum pressure value of the working pressure range are determined, as will be described below.

In the normal insufflating mode the insufflator 100 delivers or withdraws insufflating gas to or from the cavity 3 through a trocar 106a similar to the trocar 6 described with reference to FIG. 1, for maintaining the cavity insufflated during the minimally invasive surgical or investigative procedure at the selected working pressure value, or the selected working volume value. Additionally, in the normal insufflating mode the pressure or volume to which the cavity 3 is being insufflated may be altered during the carrying out of the surgical or investigative procedure.

In the set-up mode of the insufflator 100 a pressure/volume relationship between the pressure in the cavity 3 (cavity pressure) and the volume thereof is determined in a substantially similar manner as already described with reference to the insufflator 1, and a graphical relationship between the cavity pressure and the cumulative volume of insufflating gas delivered to the cavity 3 from the commencement of insufflating of the cavity 3 in the set-up mode is illustrated in FIG. 4. The minimum working pressure value and the optimum maximum pressure value of the working pressure range are determined from the pressure/volume relationship illustrated in FIG. 4, as will be described below. Before describing the operation of the insufflator 100 in the set-up mode and in the normal insufflating mode, the insufflator 100 and its connection to the body 5 of the subject will first be described.

The trocar 106a comprises an instrument channel 108 extending therethrough from a proximal end 109 to a distal end 110, which extends into and communicates with the cavity 3. The trocar 106a comprises an insufflating gas inlet port 112 through which insufflating gas is delivered from the insufflator 100 to the trocar 106a, and in turn to the cavity 3 through a bore (not shown) extending along the wall of the trocar 106a from the inlet port 112 to the distal end 110 of the trocar 106a. Alternatively, if the trocar is not provided with such an insufflating gas port, the insufflating gas may be delivered through a Veress needle into the cavity 3 or through a conduit extending into the cavity 3 through the instrument channel 108 of the trocar 106a or through an instrument channel of another trocar, for example, a trocar 106b.

The insufflator 100 comprises a housing 114, which may include a source of insufflating gas, which if included in the housing 114 would normally be provided in a pressurised container containing pressurised insufflating gas, typically, carbon dioxide. However, in this embodiment of the invention the insufflator 100 is adapted to receive the pressurised insufflating gas from an external pressurised insufflating gas source 115, which typically, comprises compressed carbon dioxide, typically, from a source of compressed carbon dioxide available in a hospital operating theatre, or in other locations in which the insufflator 100 is being used. An insufflating gas inlet port 117 is provided in the housing 114 for connecting the insufflator 100 to the external pressurised insufflating gas source 115.

The insufflator 100 is adapted for coupling to a vacuum system 119, in this embodiment of the invention an external vacuum system 119, such as vacuum system in a hospital operating theatre or in other locations in which the insufflator 100 may be used. A vacuum port 120 is provided in the housing 114 for connecting the insufflator 100 to the external vacuum system 119. However, it is envisaged that in some embodiments of the invention the insufflator 100 may be provided with an internal vacuum system, which typically, would comprise a vacuum pump located within the housing 114 or a Venturi vacuum generating system which would also be located in the housing 114. If the vacuum system were provided as a Venturi vacuum generating system, the Venturi vacuum generating system typically would be supplied with pressurised insufflating gas from the pressurised insufflating gas source 115 for generating the vacuum.

A signal processor, in this case provided by a microprocessor 122 located in the housing 114 is programmed to control the operation of the insufflator 100 as will be described below. However, it will be appreciated that any other suitable form of signal processor may be provided, for example, a microcontroller, a programable logic controller, or any other suitable signal processor.

A flow control means for controlling the flow of insufflating gas to and from the cavity 3, in this embodiment of the invention comprises a flow controller 124 and an isolating valve 125, both of which are located in the housing 114 and are operated under the control of the microprocessor 122. The flow controller 124 is connected to the insufflating gas inlet port 117 for receiving the insufflating gas therethrough from the pressurised insufflating gas source 115. The flow controller 124 is operated under the control of the microprocessor 122 for controlling the flow of insufflating gas to the cavity 3 for in turn controlling the pressure in the cavity 3, as will be described below, for maintaining the pressure and/or the volume of the cavity 3 at the selected working pressure value or the selected working volume value thereof.

A two-way valve 127, in this embodiment of the invention a solenoid operated two-way valve is located in the housing 114, and comprises first and second inlet ports 128 and 129, and an outlet port 130. The two-way valve 127 is selectively and alternately operable under the control of the microprocessor 122 in a first state with the outlet port 130 communicating with the first inlet port 128 and isolated from the second inlet port 129, and in a second state with the outlet port 130 communicating with the second inlet port 129 and isolated from the first inlet port 128. The outlet port 130 of the two-way valve 127 is connected to an outlet port 132 in the housing 114. The flow controller 124 is connected to the first inlet port 128 of the two-way valve 127 through a flow sensor 135 for monitoring the flow of insufflating gas therethrough, as will be described below. The outlet port 132 from the housing 114 is adapted for connecting to a conduit 134, which extends from the outlet port 132 to the insufflating gas inlet port 112 of the trocar 106a and is connected thereto, so that when the two-way valve 127 is in the first state thereof, the flow controller 124 is connected to the outlet port 132 for delivering insufflating gas to the cavity 3 therethrough and through the conduit 134 to the cavity 3 for maintaining the cavity 3 insufflated at the selected working pressure value or the selected working volume value, as will be described below.

The isolating valve 125 comprises solenoid operated isolating valve, and is connected between and to the vacuum port 120 and the second inlet port 129 of the two-way valve 127. The isolating valve 125 is selectively and alternately operable under the control of the microprocessor 122 in an isolating state isolating the second inlet port 129 of the two-way valve 127 from the vacuum port 120, and an open state communicating the second inlet port 129 of the two-way valve 127 with the vacuum port 120 for applying a vacuum to the outlet port 132 of the insufflator 100 when the two-way valve 127 is in the second state for applying vacuum to the cavity 3 through the conduit 134, for in turn drawing insufflating gas therefrom in order to reduce the cavity pressure to the selected working pressure value, or if a working volume value had been selected to reduce the volume of the cavity to the selected working volume value.

The flow sensor 135 located in the housing 114 between the flow controller 124 and the first inlet port 128 of the two-way valve 127 comprises a flow rate sensor 135 for monitoring the flow rate of the insufflating gas therethrough from the flow controller 124 to the cavity 3. The flow rate sensor 135 produces a signal indicative of the rate at which the insufflating gas is being delivered to the cavity 3 by the flow controller 124. The microprocessor 122 is programmed to read the signal produced by the flow rate sensor 135 and to compute the cumulative volume of insufflating gas delivered to the cavity 3 from the commencement of delivery of insufflating gas thereto when the insufflator 100 is operating in the set-up mode, as will be described below.

A pressure sensor 137 located in the housing 114 is connected to the outlet port 132 from the insufflator 100 between the outlet port 130 of the two-way valve 127 and the outlet port 132 for monitoring the pressure in the cavity 3 through the conduit 134. The pressure sensor 137 produces a signal indicative of the cavity pressure which is read by the microprocessor 122. Alternatively, instead of locating the pressure sensor 137 in the housing 114, a pressure sensor may be located in the cavity 3. For example, a pressure sensor may be located on the trocar 106a or the trocar 106b adjacent the distal end 110 on the outer surface thereof, and the signal from the pressure sensor indicative of the pressure in the cavity 3 would be communicated to the microprocessor 122 either wirelessly or through a wire extending from the pressure sensor through the trocar 106a or 106b as the case may be, to the microprocessor 122.

An interface means, namely, an interface 140, which may comprise a touch screen, a keypad or any other suitable interface, and which in this case comprises a touch screen 141, is located on the housing 114, and acts as a main input means to enable entry into the microprocessor 122 of the selected working pressure value or the selected working volume value, at which the cavity is to be insufflated when the insufflator 100 is operating in the normal insufflating mode, and other relevant data, for example, the maximum safe pressure value to which the cavity 3 may be insufflated in the normal insufflating mode and also in the set-up mode. The interface 140 also comprises a pair of button operated switches, namely, a set-up mode select button operated switch 142 and a normal insufflating run mode select button operated switch 143 for selecting the operating modes of the insufflator 100, namely, the set-up mode or the normal insufflating mode, respectively. The microprocessor 122 is programmed to read signals from the touch screen 141 and from the set-up mode and normal insufflating run mode select button operated switches 142 and 143 of the interface 140.

An electronic storing means comprising an electronic memory 145, which may be any suitable electronic memory such as a random access memory, is located in the housing 114, and is accessible to the microprocessor 122 for storing data for access by the microprocessor 122 as will be described below. In some embodiments of the invention the memory 145 may be provided by a memory of the microprocessor 122.

The microprocessor 122 controls a visual display screen 147 located on the housing 114 which provides visual data to a surgeon or clinician regarding the operation of the insufflator 100 including the current pressure and/or volume at which the cavity 3 is being insufflated.

An alerting device for producing an alerting signal is also operated under the control of the microprocessor 122 in the event of the cavity pressure exceeding either of a maximum safe pressure value, or the optimum maximum pressure value which is determined by the microprocessor 122 when the insufflator is operating in the set-up mode, as will be described below. The alerting device, in this case comprises both an alarm sounder 148 and a warning light 149, both of which are mounted on the housing 14 for producing an audible alerting signal and a visual alerting signal, respectively.

Initially, the insufflator 100 is operated in the set-up mode to determine the minimum working pressure value and the optimum maximum pressure value of the working pressure range of the cavity 3 of the subject. The insufflator 100 is operated in the set-up mode by depressing the set-up mode select button operated switch 142 in the interface 140. In the set-up mode, the working pressure range is determined from the pressure/volume relationship between the cavity pressure and the volume of the cavity. The volume of the cavity 3 is determined from the cumulative volume of insufflating gas delivered to the cavity 3 from the commencement of delivery of insufflating gas to the cavity in the set-up mode. The volume of the cavity 3 approximates to the volume of insufflating gas delivered to the cavity in the set-up mode. The working pressure range of the cavity 3 is determined from a part of the pressure/volume relationship between the cavity pressure and the cumulative volume of insufflating gas delivered to the cavity during which the pressure/volume relationship is substantially linear and the increase in cavity pressure per unit volume of insufflating gas delivered to the cavity is substantially constant from the minimum working pressure value to the optimum maximum pressure value.

The optimum maximum pressure value of the working pressure range is determined in a similar manner to that in which the optimum maximum pressure value for the cavity 3 described with reference to FIGS. 1 and 2, and is the cavity pressure above which the increase in the volume of the cavity 3 per unit increase in the cavity pressure transitions from a first substantially constant value to a second substantially constant value at which the increase in volume of the cavity 3 per unit increase in the cavity pressure is reducing or is minimal. Or put another way, the optimum maximum pressure value is the cavity pressure at which the pressure/volume relationship between the cavity pressure and the volume of the cavity 3, illustrated in FIG. 4 and described below, transitions from the first pressure/volume relationship to the second pressure/volume relationship.

The minimum working pressure value of the working pressure range is determined as the minimum pressure at which the cavity pressure commences to increase after the commencement of delivery of insufflating gas to the cavity 3. As discussed with reference to FIG. 2, during the initial pressure/volume relationship between cavity pressure and the volume of the cavity 3 from the commencement of delivery of insufflating gas to the cavity 3 as insufflating gas is delivered to the cavity 3, the pressure in the cavity remains substantially constant until the first pressure/volume relationship commences when the cavity pressure per unit of insufflating gas delivered to the cavity commences to increase. The minimum working pressure value of the working pressure range is determined as the value of the cavity pressure as the pressure/volume relationship between the cavity pressure and the volume of the cavity 3 transitions from the initial pressure/volume relationship to the first pressure/volume relationship.

Referring now to FIG. 4, a typical pressure/volume relationship, which is substantially similar to that illustrated in FIG. 2, between actual cavity pressure and the actual cumulative volume of insufflating gas delivered to a peritoneal cavity of a subject from the commencement of insufflating of the cavity is illustrated graphically by a graph 150 of FIG. 4. In FIG. 4, the cavity pressure is plotted on the ordinate, namely, the Y-axis, and the cumulative volume of insufflating gas delivered to the cavity is plotted on the abscissa, namely, the X-axis from the commencement of insufflating of the cavity. A graph 153 represents the pressure/volume relationship between the cavity pressure and the cumulative volume of insufflating gas delivered to the peritoneal cavity of the subject after a smoothing algorithm has been applied to the actual pressure/volume relationship represented by the graph 150.

As can be seen from the graphs 150 and 153, during the initial pressure/volume relationship from the commencement of delivery of insufflating gas to the cavity, as insufflating gas is being delivered to the cavity, the pressure of the cavity remains substantially constant as the cumulative volume of the insufflating gas which is delivered to the cavity increases, until the cavity has been filled with the insufflating gas. At this point, namely, at the point A of the graphs 150 and 153, as further insufflating gas is delivered to the cavity, the cavity pressure commences to rise in the first pressure/volume relationship. From point A of the graph 150 during the first pressure/volume relationship as further insufflating gas is delivered into the cavity, the increase in cavity pressure per unit volume of insufflating gas delivered to the cavity, is substantially constant until point B of the graph 150. Accordingly, the first pressure/volume relationship between point A and point B of the graph 150 is a substantially linear first pressure/volume relationship.

At point B of the graph 150, the first pressure/volume relationship transitions to an intermediate pressure/volume relationship, which is non-linear, and during which the increase in the pressure in the cavity per unit volume of insufflating gas delivered to the cavity progressively increases. The non-linear intermediate pressure/volume relationship continues to point C of the graph 150. At point C of the graph 150, the non-linear intermediate pressure/volume relationship transitions to a second pressure/volume relationship, which comprises a substantially linear relationship with the increase in cavity pressure per unit volume of insufflating gas delivered to the cavity being substantially constant. However, the increase in cavity pressure per unit volume of insufflating gas delivered to the cavity 3 is substantially greater during the second pressure/volume relationship than during the first pressure/volume relationship.

From point B of the graph 150 at the end of the first pressure/volume relationship, the cavity pressure commences to rise from a first pressure value P₁ at a non-linear ever-increasing rate per unit volume of insufflating gas delivered to the cavity to a second pressure value P₂ at the point C of the graph 150 at the commencement of the second pressure/volume relationship. Accordingly, the gain in working volume in the cavity per unit volume of gas delivered to the cavity begins to decrease from the point B, while the increase in pressure per unit volume of insufflating gas delivered to the cavity begins to increase. Thus, at some point between the first pressure value P₁ at point B of the graph 150 and the second pressure value P₂ at point C of the graph 150, and onward from the point C, the gain in working volume in the cavity per unit volume of gas delivered to the cavity is marginal while the cavity pressure per unit volume of gas delivered to the cavity commences to rapidly increase.

The microprocessor 122 is programmed in the set-up mode to determine the working pressure range, and in turn the working volume range as being represented by the linear first pressure/volume relationship from the smoothed graph 153 from the minimum working pressure value Pₘᵢₙ at the point A of the smoothed graph 153 to the point 155 on the smoothed graph 153, which defines the optimum maximum pressure value Pₘₐₓ. The first pressure/volume relationship is represented by the portion 156 of the smoothed pressure/volume relationship of graph 153 between the point A and the point 155, and the second pressure/volume relationship is represented by the portion 157 of the smoothed pressure/volume relationship of the graph 153 from the point 155 onwards. The minimum working pressure value Pₘᵢₙ of the working pressure range is determined as the first point of inflection, namely, point A of the smoothed graph 153, at which the cavity pressure commences to rise at the end of the initial pressure/volume relationship. The optimum maximum pressure value Pₘₐₓ of the working pressure range is determined as being the second point of inflection of the smoothed graph 153, namely, the point 155 of intersection of the portion 156 of the smoothed graph 153 representing the first smoothed pressure/volume relationship, and the portion 157 of the smoothed graph 153 representing the second smoothed pressure/volume relationship.

Typically, depending on the sex, age and the body mass index of a subject, the value of the minimum working pressure value Pₘᵢₙ of the working pressure range would lie in the range of 7 mmHg to 10 mmHg, and the optimum maximum cavity pressure value Pₘₐₓ of the working pressure range would lie in the range of 10 mmHg to 20 mmHg.

Returning now to the operation of the insufflator 100 in the set-up mode, while the insufflator 100 is operating in the set-up mode, the microprocessor 122 controls the flow controller 124 to deliver insufflating gas to the cavity 3 of the subject at a substantially constant rate, typically, at a rate in the range of 0.5 litres per minute to 5 litres per minute, and reads the values of the signal from the flow rate sensor 135 and the values of the signal from the pressure sensor 137 at predefined time intervals, in this case, predefined time intervals of approximately 1 second to 1.5 seconds as the insufflating gas is being delivered to the cavity 3. At the end of each predefined time interval, the flow controller 124 is operated by the microprocessor 122 to isolate the cavity 3 from the insufflating gas source 115 for a time period of approximately 0.5 seconds to allow the cavity pressure to stabilise for monitoring thereof by the pressure sensor 137. At the end of each time period of 0.5 seconds, the flow controller 124 is operated by the microprocessor 122 for the next predefined time interval to again deliver insufflating gas to the cavity 3.

As the values of the signals are read from the flow rate sensor 135 and the pressure sensor 137 at the end of each predefined time interval, the microprocessor 122 is programmed to determine the cumulative volume of insufflating gas delivered to the cavity 3 from the commencement of delivery of the insufflating gas to the cavity 3 and the corresponding cavity pressure value. The determined cumulative volumes of insufflating gas delivered to the cavity 3 and the corresponding cavity pressure values are time-stamped, cross-referenced with each other and stored in the memory 145. The microprocessor 122 continues to deliver insufflating gas to the cavity 3 and to read the signals from the flow rate sensor 135 and from the pressure sensor 137 until the cavity pressure reaches the maximum safe pressure value, at which stage insufflating of the cavity is terminated. The microprocessor 122 is programmed to then compute a smoothed pressure/volume relationship between the cavity pressure and the cumulative volume of insufflating gas delivered to the cavity 3 from the stored and cross-referenced values of the cumulative volumes of the insufflating gas and the corresponding pressure values. The smoothing is carried out by applying a suitable smoothing algorithm, in this case a moving average algorithm to the stored and cross-referenced values. The microprocessor 122 is programmed to also compute a smoothed value of the increase in the cavity pressure per unit volume of insufflating gas delivered to the cavity 3 from each pair of the stored and cross-reference values of the cavity pressure and the cumulative volumes of the insufflating gas, which essentially give the slope of the smoothed graph 153 in order to determine the first and second points of inflection of the smoothed pressure/volume relationship, which correspond to the points A and 155, respectively, on the smoothed graph 153 of FIG. 4.

On determining the first and second points of inflection on the pressure/volume relationship, the microprocessor 22 is programmed to determine the minimum working pressure value Pₘᵢₙ of the working pressure range as the cavity pressure at the first point of inflection, namely, at the point A on the smoothed graph 153. In other words, the value of the minimum working pressure value Pₘᵢₙ is the value of the cavity pressure at which the cavity pressure commences to increase after the commencement of insufflating of the cavity 3.

The microprocessor 122 is programmed to then determine the optimum maximum pressure value Pₘₐₓ of the working pressure range as the cavity pressure at the second point of inflection of the smoothed pressure/volume relationship corresponding to the point 155 of the smoothed graph 153, where the first smoothed pressure/volume relationship 156 transitions to the second smoothed pressure/volume relationship 157. Alternatively, the microprocessor 122 may be programmed to determine the value of the optimum maximum pressure value Pₘₐₓ of the working pressure range as the point of intersection 155 of the portions 156 and 157 of the smoothed graph 153 by either interpolating between the two portions 156 and 157 or by extrapolating the portion 156 forwardly beyond the value of the first pressure P₁, and by extrapolating the portion 157 backwards from the value of the second pressure P₂.

On determining the values of the minimum working pressure Pₘᵢₙ and the optimum maximum pressure Pₘₐₓ, the minimum working pressure value Pₘᵢₙ and the optimum maximum pressure value Pₘₐₓ are stored in the memory 145, and the microprocessor 122 outputs a signal indicative of the values of the minimum working pressure Pₘᵢₙ and the optimum maximum pressure Pₘₐₓ for display on the visual display screen 147.

Additionally, the microprocessor 122 may be programmed, when operating the insufflator 100 in the normal insufflating mode, to control the flow controller 124 to prevent the cavity pressure exceeding the optimum maximum pressure value Pₘₐₓ.

On the minimum working pressure value and the optimum maximum pressure value being determined, the microprocessor 122 is programmed to prepare a look-up table 160 as illustrated in FIG. 5 comprising a first column 161 in which selectable working pressure values within the working pressure range are stored, and a second column 162 in which values indicative of corresponding selectable working volumes of the cavity 3 within the working volume range are stored and cross-referenced with the corresponding working pressure values in the first column 161. In the first row 163a of the look-up table 160 the selectable optimum maximum pressure value Pₘₐₓ is stored in the first column 161, and the value indicative of the maximum selectable working volume Vₘₐₓ of the working volume range is stored in the second column 162 corresponding to the optimum maximum pressure value Pₘₐₓ. The value of the maximum cavity volume Vₘₐₓ is computed by the microprocessor 122 based on the cumulative value of the insufflating gas delivered to the cavity 3 from the commencement of insufflating of the cavity 3 until the cavity pressure reached the optimum maximum pressure value with the cumulative volume of the insufflating gas corrected for cavity pressure.

It is envisaged that in some embodiments of the invention the actual maximum cavity volume Vₘₐₓ may not be computed, and instead, a representation of the maximum volume would be included in the look-up table, for example, a representation similar to the representation “Vₘₐₓ”.

In the bottom or last row 163n of the look-up table 160, the value of the minimum working pressure Pₘᵢₙ of the working pressure range is stored in the first column 162, and the value of the minimum working volume, namely, Vₘᵢₙ of the working volume range of the cavity 3 is stored in the second column 162 corresponding to the minimum working pressure value Pₘᵢₙ. As discussed above, in some embodiments of the invention it is envisaged that the value of the minimum working volume would not be computed, and a representation of the minimum working volume would be included in the look-up table similar to the representation “Vₘᵢₙ” in the look-up table 160 of FIG. 5.

In the intervening rows 63b to 63n−1 of the look-up table 60, values of the intervening selectable working pressures of the working pressure range between the minimum working pressure value Pₘᵢₙ and the optimum maximum pressure value Pₘₐₓ are stored in column 161 of the look-up table 160. The intervening selectable working pressure values are selected and stored in decremented values of 5% of the optimum maximum pressure value. It will however be readily apparent to those skilled in the art that the decremented values of the intervening selectable working pressure values may be greater or less than 5% of the optimum maximum pressure value. Indeed, in some embodiments of the invention it is envisaged that the decremented values of the intervening selectable working pressure values may be as large as 25% of the optimum maximum pressure value. The corresponding intervening selectable working volumes corresponding to the intervening selectable working pressure values are stored in the second column 162 of the look-up table 160 in the corresponding intervening rows 63b to 63n−1 in corresponding decrements of 5% of the maximum working volume Vₘₐₓ.

With the look-up table 160 completed, the microprocessor 122 displays the working pressure range of the selectable working pressure values from the minimum working pressure value Pₘᵢₙ to the optimum maximum pressure value Pₘₐₓ as stored in the look-up table 160 on the touch screen 141 of the interface 140, and also displays the working volume range of the selectable working volumes from the selectable minimum working volume Vₘᵢₙ to the maximum working volume Vₘₐₓ as stored in the look-up table 160 on the touch screen 141 of the interface 140, thereby completing operation of the insufflator 100 in the set-up mode. With the selectable working pressure values and the selectable working volumes displayed on the touch screen 141, a surgeon or clinician may select the desired selectable working pressure value or the selectable working volume value at which the cavity 3 is to be insufflated from the touch screen by touching the appropriate displayed working pressure value or the appropriate displayed working volume value on the touch screen 141.

To operate the insufflator in the normal insufflating mode, the normal insufflating run mode select button operated switch 143 in the interface 140 is depressed. The surgeon then selects and enters the working pressure value or the working volume value at which the cavity 3 is to be insufflated by selecting the relevant working pressure value or the working volume value on the touch screen 141. The microprocessor 122 reads the selected working pressure or volume value from the touch screen 141. The surgeon or clinician depresses the normal insufflating run mode select button operated switch 143 a second time to activate the insufflator 100 to commence insufflating the cavity 3. The microprocessor 122 operates the two-way valve 127 in the first state to connect the flow controller 124 through to the outlet port 132 of the insufflator 100, and operates the flow controller 124 in response to the signal read from the pressure sensor 137 to insufflate the cavity 3 to the selected working pressure value read from the touch screen 141 or corresponding to the selected working volume value read from the touch screen 141, as the case may be, and maintains the cavity pressure at the selected working pressure value. If the working volume value is selected, the microprocessor 122 operates the flow controller 124 to maintain the cavity pressure at the working pressure value corresponding to the selected working volume value in order to maintain the cavity 3 at the selected working volume value. The current cavity pressure and the current volume of the cavity 3 are continuously displayed on the visual display screen 147, the cavity pressure being displayed as the actual cavity pressure, and the volume of the cavity 3 being displayed as a percentage of the maximum working volume value Vₘₐₓ, or graphically.

During the procedure should the surgeon or clinician wish to alter the pressure or the volume at which the cavity 3 is being insufflated, the surgeon or clinician selects and enters the desired working pressure value or the desired working volume value through the touch screen 141. The microprocessor 122 reads the selected working pressure value or the selected working volume value from the touch screen 141. If the selected working pressure value is greater than the current cavity pressure at which the insufflator 100 is insufflating the cavity 3, or if the selected working pressure value is greater than the current cavity pressure at which the cavity 3 is being insufflated to maintain the cavity at the current volume, the microprocessor 122 operates the flow controller 124 to increase the rate at which insufflating gas is being delivered to the cavity 3 to increase the cavity pressure to the selected working pressure value read from the touch screen 141, or to increase the cavity pressure to the working pressure value corresponding to the selected working volume value as the case may be. The microprocessor 122 controls the operation of the flow controller 124 in response to the signal from the pressure sensor 137 for maintaining the cavity pressure at the new working pressure value until the surgeon or clinician selects another working pressure value or another working volume value to which the cavity 3 is to be insufflated, or until the procedure has been completed.

On the other hand, should the selected working pressure value or the selected working volume value be less than the current cavity pressure or the current volume at which the cavity 3 is being insufflated, if the difference between the selected working pressure or volume value and the current cavity pressure or volume is relatively small, and the selected working pressure or volume could be reached by normal leakage of insufflating gas from the cavity 3, the microprocessor 122 is programmed to operate the flow controller 124 to reduce the rate at which the insufflating gas is being delivered to the cavity 3, or to temporarily pause delivery of insufflating gas to the cavity 3, until the cavity pressure falls to the selected working pressure value or to the working pressure value corresponding to the selected working volume value. Once the cavity pressure has fallen to the selected working pressure value or the working pressure value corresponding to the selected working volume value, the microprocessor 122 operates the flow controller 124, in response to the signal read from the pressure sensor 137 indicative of the cavity pressure, to maintain the cavity pressure at the selected working pressure value or the working pressure value corresponding to the selected working volume value.

If the selected working pressure value is significantly less than the current cavity pressure at which the cavity 3 is being insufflated, or if the working pressure value corresponding to the selected working volume value is significantly less than the current cavity pressure at which the cavity is being insufflated, the microprocessor 122 operates the two-way valve 127 in the second state with the second inlet port 129 connected to the outlet port 130 and operates the isolating valve 125 from the isolating state to the open state thereby connecting the cavity 3 to the vacuum system 119 for drawing insufflating gas from the cavity to reduce the cavity pressure to the selected working pressure value or to the working pressure value corresponding to the selected working volume value, as the case may be. On the cavity pressure falling to the selected working pressure value or the working pressure value corresponding to the selected working volume value, the microprocessor 122 operates the isolating valve 125 into the isolating state and operates the two-way valve 127 into the first state. The microprocessor 122 then operates the flow controller 124 in response to the cavity pressure read from the signal from the pressure sensor 137, to maintain the cavity pressure at the selected working pressure value or the working pressure value corresponding to the selected working volume value.

During operation of the flow controller 124 by the microprocessor 122 to insufflate the cavity 3, the microprocessor 122 operates the flow controller to deliver the insufflating gas to the cavity 3 during sequential first predefined time periods each of approximately 1.5 seconds duration, and between the respective first predefined time periods, the microprocessor 122 operates the flow controller 124 for second predefined time periods of approximately 0.5 seconds during which the flow controller 124 is operated to isolate the cavity 3 from the insufflating gas source 115 in order that the signal read from the pressure sensor 137 during each second predefined time period is indicative of the cavity pressure, so that the cavity pressure may be accurately determined from the signal read from the pressure sensor 137. Additionally, when the isolating valve 125 is operated under the control of the microprocessor 122 to apply vacuum to the cavity 3, the isolating valve 125 is operated by the microprocessor 122 sequentially for similar first predefined time periods, and between the respective predefined time periods, the isolating valve 125 is operated into the isolating state to isolate the cavity 3 from the vacuum system 119, so that the signal read from the pressure sensor 137 by the microprocessor 122 during the second time periods is indicative of the cavity pressure in the cavity 3 of the subject.

During operation of the insufflator 100, should the cavity pressure exceed the optimum maximum pressure value, or should the cavity pressure exceed the maximum safe pressure value, the microprocessor 122, in response to the signal from the pressure sensor being indicative of either the cavity pressure exceeding the optimum maximum pressure value or the maximum safe pressure value, activates the alarm sounder 148 and the warning light 149 to alert the surgeon or clinician to the excess cavity pressure. The maximum safe pressure value may be preset in the insufflator 100 or may be entered into the microprocessor 122 of the insufflator 100 by the surgeon or clinician through, for example, the touch screen 141.

Referring now to FIG. 6, there is illustrated an insufflator according to another embodiment of the invention indicated generally by the reference numeral 170. The insufflator 170 is substantially similar to the insufflator 100 and similar components are identified by the same reference numerals. In this embodiment of the invention the insufflator 170 is operable in a set-up mode substantially similar to that of the insufflator 100 and in a normal operating mode also substantially similar to that of the insufflator 100. In the set-up mode the insufflator 170 determines the working pressure range only of selectable working pressure values to which a cavity 3 of a subject 5 may be insufflated. Like the insufflator 100, the selectable working pressure values range from the optimum maximum pressure value Pₘₐₓ to the minimum working pressure value Pₘᵢₙ in decrements of 5% of the optimum maximum pressure value. In the normal insufflating mode, the insufflator 170 is operated for insufflating the cavity 3 at the selected one of the selectable working pressure values.

The only differences between the insufflator 170 and the insufflator 100 are that the isolating valve 125 has been replaced by a vacuum pump 171, and the interface means, as well as comprising the main input means, namely, the touch screen 141, also comprises a secondary input means. In this embodiment of the invention the secondary input means comprises first and second pedal operated switches 172 and 173, respectively, for inputting signals to the microprocessor 122 to increase or decrease the cavity pressure from the current working pressure value at which the cavity 3 is being insufflated to a higher or lower selectable working pressure value, as will be described below.

The vacuum pump 171 is located in the housing 114 of the insufflator 170 and is connected to the second inlet port 129 of the two-way valve 127. The vacuum pump 171 is operated under the control of the microprocessor 122 for reducing the cavity pressure in the event of the selected working pressure value being less than the current cavity pressure, as will be described below. When the vacuum pump 171 is being operated by the microprocessor 122 to apply a vacuum to the cavity 3, the two-way valve 127 is operated into the second state for connecting the vacuum pump 171 through the outlet port 32 of the insufflator 170 and in turn through the conduit 134 to the cavity 3.

Turning now to the first and second foot pedal operated switches 172 and 173, the first and second foot pedal operated switches comprise mono-stable, bi-state electrical switches 174 and 175, respectively, operable in a stable normally open circuit state and an unstable closed circuit state. Foot pedals 176 and 177 operate the electrical switches 174 and 175, respectively, from the open circuit state to the closed circuit state. The first foot pedal operated switch 172 is configured so that each time the first foot pedal switch 172 is operated by depressing foot pedal 176 once, the corresponding electrical switch 174 is operated to apply a single signal to the microprocessor 122, and on receiving each signal from the electrical switch 174 of the first foot pedal operated switch 172, the microprocessor 122 operates the flow controller 124 to increase the flow of insufflating gas to the cavity 3 to increase the cavity pressure by one increment of working pressure, namely, 5% of the optimum maximum pressure value above the working pressure value at which the cavity is currently being insufflated, since in this embodiment of the invention the selectable working pressure values of the working pressure range are selectable in increments of 5% of the optimum maximum pressure value Pₘₐₓ from the minimum working pressure value Pₘᵢₙ to the optimum maximum pressure value Pₘₐₓ. If the foot pedal 176 of the first foot pedal switch 172 is depressed a plurality of times sequentially to operate the corresponding electrical switch 174 to sequentially produce a corresponding number of signals, the microprocessor 122 counts the number of sequential signals produced by the corresponding electrical switch 174, and operates the flow controller 124 to increase the flow of insufflating gas to the cavity 3 in order to increase the cavity pressure by the number of working pressure increments above the working pressure value at which the cavity is currently being insufflated corresponding to the number of sequential signals produced by the electrical switch 174. When the cavity pressure has been increased to the selected cavity pressure, the microprocessor 122 operates the flow controller 124 in response to the signal read from the pressure sensor 137 to maintain the cavity pressure at the now selected working pressure value.

The second foot pedal operated switch 173 is configured so that each time the corresponding electrical switch 175 is operated by depressing the foot pedal 177, a signal is applied to the microprocessor 122. On receiving each signal from the second foot pedal operated switch 173, the microprocessor 122 operates the flow controller 124 to reduce the delivery rate of insufflating gas to the cavity, or to pause supply of insufflating gas to the cavity until the cavity pressure falls by one increment of working pressure from the working pressure value at which the cavity is currently being insufflated. However, if the foot pedal 177 of the second foot pedal switch 173 is depressed a plurality of times sequentially to operate the corresponding electrical switch 175 to sequentially produce a corresponding number of signals, the microprocessor 122 counts the number of signals produced by the electrical switch 175 of the second foot pedal switch 173 and operates the vacuum pump 171 to generate a vacuum and operates the two-way valve 127 into the second state to apply the vacuum to the cavity 3 to reduce the cavity pressure by the number of working pressure increments below the working pressure value at which the cavity is currently being insufflated corresponding to the number of sequential signals produced by the electrical switch 175. When the cavity pressure has been reduced to the selected cavity pressure value, the microprocessor 122 deactivates the vacuum pump 171, operates the two-way valve 127 into the first state, and operates the flow controller 124 in response to the signal read from the pressure sensor 137 to maintain the cavity pressure at the now selected working pressure value.

In this embodiment of the invention the insufflator 170, also displays the selectable pressures of the working pressure range on the touch screen 141, so that the pressure at which the cavity 3 is to be insufflated may also be selected from the touch screen 141 by touching the appropriate displayed pressure value on the touch screen at which the cavity 3 is to be insufflated.

Otherwise, the insufflator 170 is similar to the insufflator 100, and its operation in both the set-up mode and in the normal insufflating mode is likewise similar to that of the insufflator 100, with the exception that in the insufflator 170 only the pressures at which the cavity may be insufflated are selectable.

Referring now to FIG. 7 there is illustrated an interface means for an insufflator according to another embodiment of the invention. The insufflator of this embodiment of the invention is substantially similar to the insufflator 170 of FIG. 6. The only difference between the insufflator according to this embodiment of the invention and the insufflator 170 lies in the interface means and in its operation under the control of the microprocessor. Accordingly, for convenience in describing the interface means of the insufflator of this embodiment of the invention, reference will be made to the insufflator 170 and the components thereof will be referred to by the same reference numerals as those of the insufflator 170.

In this embodiment of the invention the interface means comprises a touch screen 180 which acts as the main input means for inputting data into the microprocessor 122. However, in this embodiment of the invention the microprocessor 122 is programmed to display a representation 182 of a part of the pressure/volume relationship between the cavity pressure and the volume of the cavity 3. The part of the pressure/volume relationship displayed on the touch screen 180 comprises the first pressure/volume relationship of the smoothed pressure/volume relationship represented by the graph 153 of the smoothed pressure/volume relationship illustrated in FIG. 4 between the point A and the point 155 of the smoothed first pressure/volume relationship 153. Accordingly, the lower end point 183 of the representation 182 which corresponds to point A of the representation 153 of FIG. 4 represents the minimum working pressure value for the cavity. The upper end point 184 of the representation 182 which corresponds to the point 155 of the representation 153 of FIG. 4, represents the optimum maximum pressure value for the cavity 3. In this embodiment of the invention the scale of the pressure values on the ordinate, namely, the Y axis is increased relative to the scale of the volume values on the abscissa, namely, on the X axis, in order to more easily facilitate selection of a working pressure value at which the cavity is to be insufflated.

The touch screen 180 is configured to allow a working pressure value at which the cavity 3 is to be selected to be inputted to the microprocessor 122 through the touch screen 180, and the microprocessor 122 is programmed to read the inputted selected working pressure value from the touch screen 180.

Two indicating means, namely, a first indicating means provided by a first cursor 186, and a second indicating means provided by a second cursor 187 are displayed under the control of the signal processor on the touch screen 180. Both the first and second cursors 186 and 187 are moveable along the representation 182 of the first pressure/volume relationship to identify pressure values on the representation 182 of the first pressure/volume relationship. The first cursor 186 is moveable along the representation 182 by the microprocessor, in response to the signal read from the pressure sensor 137 indicative of the cavity pressure, and is located on the representation 182 by the microprocessor 122 to indicate the current cavity pressure of the cavity. The second cursor 187 indicates the selected working pressure value at which the cavity 3 is to be insufflated. The second cursor 187 is moveable by appropriately touching the touch screen 180 or by the microprocessor 122 in response to an input signal from an input means. In this embodiment of the invention the first cursor 186 and the second cursor 187 are provided by respective circle cursors which encircles the current cavity pressure on the representation 182 in the case of the first cursor 186, and in the case of the second cursor 187 encircles the cavity pressure value on the representation 182 of the first pressure/volume relationship corresponding to the working pressure value at which the cavity is to be insufflated.

To select a working pressure value at which the cavity 3 is to be insufflated through the touch screen 180, a surgeon or clinician touches the second cursor 187 on the touch screen 180 and moves the second cursor 187 along the representation 182 of the first pressure/volume relationship to a location on the representation 182 corresponding to the cavity pressure at which the cavity is to be insufflated. Alternatively, the surgeon or clinician may touch the representation 182 of the first pressure/volume relationship on the touch screen 180 at the location of the cavity pressure corresponding to the working pressure value at which the cavity 3 is to be insufflated, and the microprocessor 122 is programmed to move the second cursor 187 to the touched location on the representation 182 of the first pressure/volume relationship.

Turning now to the selection of the working pressure value at which the cavity is to be insufflated by an input signal from an input means, in this embodiment of the invention the input means comprises a secondary input means provided by the foot pedal operated first and second switches 172 and 173 of the insufflator of FIG. 6. On the microprocessor 122 detecting a signal from either one of the first or second foot pedal operated switches 172 and 173, the microprocessor 122 urges the second cursor 187 appropriately along the representation 182 of the first pressure/volume relationship. Each time the relevant one of the first or second foot pedal operated switches 172 and 173 is operated to apply the corresponding signal to the microprocessor 122, the microprocessor 122 urges the second cursor 187 along the representation 182 of the first pressure/volume relationship a distance corresponding to a number of sequential signals produced by the relevant one of the foot pedal operated switches 172 or 173, and the direction along the representation 182 of the first pressure/volume relationship the second cursor 187 is moved is determined by the one of the first and second foot pedal switches 172 and 173 which produced the signal. In this embodiment of the invention the incremental value of each incremental working pressure value is selectable and is entered into the microprocessor 122 through the touch screen during or on completion of operation of the insufflator in the set-up mode. Thus, the microprocessor 122 is programmed to increment or decrement the cavity pressure upwardly or downwardly from the working pressure value at which the cavity is currently being insufflated by one increment in response to each signal produced by the relevant one of the first and second foot pedal operated switches 172 or 173.

On the newly selected working pressure value having been entered on the touch screen 180, or through either one of the first or second foot pedal switches 172 or 173, the microprocessor 122 operates either the flow controller 124 or the vacuum pump 171 and appropriately operates the two-way valve 127 into the appropriate one of the first or second states to increase the delivery of insufflating gas to the cavity 3 to increase the cavity pressure, or to draw insufflating gas from the cavity 3 to decrease the cavity pressure, depending on the position of the second cursor 187 relative to the first cursor 186.

It is also envisaged in this embodiment of the invention that the first and second foot pedal operated switches may comprise rheostat analogue switches, or equivalent digital switches, which in the case of a rheostat switch would produce an analogue signal proportional to the degree to which the corresponding foot pedal 176 or 177 is depressed, and in which case, the microprocessor 122 would be programmed to urge the second cursor 187 along the representation 182 of the first pressure/volume relationship, the appropriate distance towards the upper end point 184 or the lower end point 183, depending on which of the first and second foot pedal operated switches 172 or 173 was operated, a distance proportional to the degree to which the foot pedal 176 or 177 of the corresponding one of the first and second foot pedal operated switch 172 or 173 is depressed. In which case, the working pressure value would be infinitely selectable between and including the minimum working pressure value represented by the lower end point 183 of the representation 182 of the first pressure/volume relationship and the upper end point 184 of the representation 182 of the first pressure/volume relationship representative of the optimum maximum pressure value.

Referring now to FIG. 8 there is illustrated an insufflating system according to the invention indicated generally by the reference numeral 200 for remotely insufflating a peritoneal cavity 3 of a subject 5 during a minimally invasive investigative or surgical procedure. The insufflating system 200 comprises an insufflator 202 located in a first location 203 adjacent the subject 5 also located in the first location 203 for insufflating the peritoneal cavity 3 of the subject 5. The insufflator 202 is operable remotely by a controller also according to the invention indicated generally by the reference numeral 205 located in a second location 206 remote from the first location 203. Typically, the first location 203 is an operating theatre of a hospital, but may be any other suitable location. The second location 206 may be in the same building as the operating theatre or the first location, but in a separate room from the operating theatre or the first location. However, in general, it is envisaged that the second location 206 will be in a different building to that of the first location 203 and may be in a different city or town to the city or town in which the first location 203 is located. Indeed, in other embodiments of the invention the second location 206 may be in a different country to the country in which the first location 203 is located.

The controller 205 communicates with the insufflator 202 through a communicating means, which in this embodiment of the invention comprises a wireless communicating means, and the communicating means comprises a first communicating means, namely, a first communications module 208 located in the insufflator 202 as will be described below, and a second communicating means comprising a second communications module 209 located in the controller 205 as will also be described below. The first and second communications modules 208 and 209 are configured for two-way communication between the controller 205 and the insufflator 202, and may be adapted for wireless communication, wired communication or for fibreoptical communication, and in some embodiments of the invention communications between the first and second communications modules 208 and 209 may be over the internet or by any other suitable communications methods.

The controller 205 enables a surgeon carrying out a minimally invasive investigative or surgical procedure remotely in the peritoneal cavity of the subject, to control the insufflator 202 remotely from the second location 206 in order to control the insufflating of the peritoneal cavity 3 of the subject 5 by the insufflator 202.

Turning initially to the insufflator 202, the insufflator 202 is substantially similar to the insufflator 170 described with reference to FIG. 6, and similar components are identified by the same reference numerals. The main difference between the insufflator 202 and the insufflator 170 is the provision of the first communications module 208 which is located in the insufflator 202 within the housing 114. The insufflator 202 like the insufflator 170 comprises a microprocessor 122 which controls the operation of the insufflator 202 in a similar manner as the microprocessor 122 controls the operation of the insufflator 170. The microprocessor 122 of the insufflator 202 reads signals received by the first communications module 208 from the second communications module 209 of the controller 205, and operates the insufflator 202 in response to the signals read from the first communications module 208. The insufflator 202 comprises the interface 140 and the touch screen 141 as well as the set-up mode select button operated switch 142 and the normal insufflating run mode select button operated switch 143 of the interface 140 similar to those of the insufflator 170 for selectively operating the insufflator 202 in the set-up mode and in the insufflating mode, respectively. The insufflator 202 comprises the first and second foot pedal operated switches 172 and 173 of the insufflator 170 for increasing and decreasing the pressure at which the cavity 3 is to be insufflated, and the insufflator 202 comprises the alarm sounder 148 and the warning light 149. Accordingly, as well as the insufflator 202 being adapted to be operated remotely under the control of the controller 205, the insufflator 202 may also be operated directly at the first location 203 if necessary or if desired.

The peritoneal cavity 3 of the subject 5 is insufflated by the insufflator 202 through a connecting conduit 134 which is connected to the outlet port 132 of the insufflator 202 and to a gas inlet port 112 of a trocar 106a which extends through the abdominal wall of the subject 5 into the peritoneal cavity 3. Typically, the insertion of the trocars 106a and 106b through the abdominal wall into the peritoneal cavity 3 of the subject 5 will be carried out by a clinician or other suitably qualified personnel in the first location. The cavity pressure is monitored by the pressure sensor 137 through the connecting conduit 134.

Turning now to the controller 205, the controller 205 comprises a housing 210. A signal processor, in this embodiment of the invention a microprocessor 212 located in the housing 210 controls the operation of the controller 205, including controlling the second communications module 209. An electronic memory 214 located in the housing 210 of the controller 205 and similar to the memory 145 of the insufflator 202 stores data relating to the subject and the insufflating thereof which is largely duplicated from the electronic memory 145 of the insufflator 202.

In order that the insufflator 202 may be fully controlled remotely by the controller 205, the controller 205 comprises an interface means provided by an interface 215 which comprises a touch screen interface 217, a set-up mode select button operated switch 219, a normal insufflating run mode select button operated switch 220, a first foot pedal operated switch 222 and a second foot pedal operated switch 223, which duplicate the interface 140, the touch screen interface 141, the set-up mode select button operated switch 142, the normal insufflating run mode select button operated switch 143 and the first and second foot pedal operated switches 172 and 173, respectively, of the insufflator 202 in order to enable the surgeon to remotely control the operation of the insufflator 202 by the controller 205. The first and second foot pedal operated switches 222 and 223 comprise normally open mono-stable, bi-state electrical switches 224 and 225 and foot pedals 226 and 227, respectively, corresponding to the electrical switches 174 and 175 and to the foot pedals 176 and 177 of the first and second foot pedal operated switches 172 and 173, respectively, of the insufflator 202. The controller 205 comprises a visual display screen 228, an alarm sounder 229 and a warning light 230 which duplicate the visual display screen 147, the alarm sounder 148 and the warning light 149 of the insufflator 202.

The second communications module 209 is controlled by the microprocessor 212 of the controller 205. The microprocessor 212 outputs signals to the second communications module 209 for transmission to and reception by the first communications module 208 in the insufflator 202, and operates the second communications module 209 to transmit the signals for reception by the first communications module 208. The signals transmitted by the second communications module 209 to the first communications module 208 comprise instructions to be read by the microprocessor 122 of the insufflator 202 to control the operation of the insufflator 202 and the insufflating of the peritoneal cavity 3 of the subject 5 in response to signals inputted into the controller 205 through the interface 215, the touch screen 217, the set-up mode and the normal insufflating run mode select button operated switches 219 and 220 and/or through the first and second foot pedal operated switches 222 and 223. The second communications module 209 of the controller 205 is configured to receive signals from the microprocessor 122 transmitted by the first communications module 208 of the insufflator 202 comprising data and/or information to be displayed on the touch screen 217 and/or the visual display screen 228 of the controller 205 similar to information and data displayed by the microprocessor 122 of the insufflator 202 on the touch screen 141 and the visual display screen 147. The second communications module 209 of the controller 205 is also configured to receive signals transmitted by the first communications module 208 from the microprocessor 122 of the insufflator 202 relating to warning signals which are read by the microprocessor 212 of the controller 205 for operating the alarm sounder 229 and/or the warning light 230, in a similar manner as the alarm sounder 148 and the warning light 149 are operated in the insufflator 202 by the microprocessor 122.

The microprocessor 212 of the controller 205 reads signals from the interface 215, the touch screen 217, the set-up mode select button operated switch 219 and the normal insufflating run mode select button operated switch 220, as well as signals inputted to the controller 205 through the foot pedal operated switches 222 and 223. The microprocessor 212 is programmed to interpret these signals inputted thereto, and to produce signals for transmission by the second communications module 209 to the first communications module 208 of the insufflator 202. The microprocessor 122 reads the signals received by the first communications module 208 from the second communications module 209 and operates the insufflator 202 in accordance with the signals read from the first communications module 208.

The microprocessor 122 of the insufflator 202 reads signals from the flow sensor 135 and the pressure sensor 137 and determines appropriate action to be taken by the insufflator 202 in insufflating the peritoneal cavity 3 of the subject 5, and displays relevant data on the touch screen 141 and the visual display screen 147 of the insufflator 202. The microprocessor 122 simultaneously transmits corresponding data through the first communications module 208 for reception by the second communications module 209 which is read by the microprocessor 212 of the controller 205. The microprocessor 212 on receiving the signals from the microprocessor 122 through the first and second communications modules 208 and 209 displays corresponding data on the touch screen 217 and/or the visual display screen 228 of the controller 205.

The insufflator 202 is operated in the set-up mode under the control of the controller 205 by operating the set-up mode select button operated switch 219 of the interface 215 of the controller 205. The microprocessor 212 of the controller 205 on detecting the signal from the set-up mode select button operate switch 219 operates the second communications module 209 to transmit a signal for reception by the first communications module 208 indicative of a request to operate the insufflator 202 in the set-up mode. The microprocessor 122 on reading the signal from the first communications module 208 indicative of the request to operate the insufflator 202 in the set-up mode, operates the insufflator 202 in the set-up mode.

In the set-up mode, the microprocessor 122 operates the flow controller to deliver insufflating gas to the peritoneal cavity 3 at a suitable constant flow rate, typically between 0.5 litres per minute and 5 litres per minute. The microprocessor 122 reads signals from the flow sensor 135 and from the pressure sensor 137 of the insufflator 202 at predefined sampling intervals. Since the cavity pressure is being monitored by the pressure sensor 137 through the connecting conduit 134, the signals from the pressure sensor 137 and from the flow sensor 135 are read at predefined sampling time intervals of approximately 1 second to 1.5 seconds. At the end of each predefined sampling time interval, the flow controller 124 is operated to isolate the cavity 3 from the insufflating gas source 115 for a stabilising time period of approximately 0.5 seconds to allow the cavity pressure to stabilise for monitoring thereof by the pressure sensor 137. At the end of each stabilising time period the signals from the pressure sensor 137 and from the flow sensor 135 are read by the microprocessor 122 and timing of the next predefined sampling time interval commences. From the signals read from the flow sensor 135 and the pressure sensor 137 the microprocessor 122 prepares the pressure/volume relationship between the cavity pressure in the peritoneal cavity 3 and the volume of the peritoneal cavity 3 as described in connection with the insufflator 100 with reference to FIGS. 3 to 5. The microprocessor 122 prepares a graphical representation of the first pressure/volume relationship between a first transition pressure value and a second transition pressure value.

The first transition pressure value is determined by the microprocessor 122 as the cavity pressure at which the initial pressure/volume relationship transitions to the first pressure/volume relationship, and the second transition pressure value is determined by the microprocessor 122 as the cavity pressure at which the first pressure/volume relationship transitions to the second pressure/volume relationship. The first transition pressure value is also the cavity pressure at the first point of inflection of the pressure/volume relationship equivalent to first point of inflection A on the smoothed graph 153 of FIG. 4 of the pressure/volume relationship between the cavity pressure and the cavity volume. The second transition pressure value is the cavity pressure at the second point of inflection of the pressure/volume relationship equivalent to the second point of inflection 155 on the smoothed graph 153 of FIG. 4 of the pressure/volume relationship between the cavity pressure and the cavity volume. The first pressure/volume relationship is digitally stored by the microprocessor 122 in the memory 145 of the insufflator 202.

Digital data representing the first pressure/volume relationship stored in the memory 145 is transmitted by the microprocessor 122 of the insufflator 202 through the first communications module 208 for reception by the second communications module 209 of the controller 205. The microprocessor 212 of the controller 205 on reading the digital data representing the first pressure/volume relationship from the second communications module 209 stores the digital data in the memory 214 of the controller 205. The microprocessor 212 displays a graphical representation of the first pressure/volume relationship on the touch screen 217 of the interface 215. The first and second transition pressure values of the range of working pressure values between which the peritoneal cavity 3 may be insufflated are displayed on the touch screen 217 and on the visual display screen 228 of the controller 205 by the microprocessor 212.

The surgeon on knowing the first and second transition pressure values of the range of working pressure values at which the peritoneal cavity 3 may be insufflated determines the maximum safe working pressure value above which the peritoneal cavity 3 should not be insufflated, and the minimum safe working pressure value below which the working pressure value in the peritoneal cavity 3 should not be allowed to fall. Typically, the maximum safe working pressure value is selected as the second transition pressure value. The minimum safe working pressure value is typically selected as a pressure value just above the first transition pressure value, and typically, a pressure value of approximately 2 mmHg to 5 mmHg above the first transition pressure value. The maximum and minimum safe working pressure values are then entered by the surgeon through the touch screen 217 of the interface 215 of the controller 205 to the microprocessor 212. The microprocessor 212 stores the maximum and minimum safe working pressure values in the memory 214, and operates the second communications module to 209 to transmit signals indicative of the maximum and minimum safe working pressure values for reception by the first communications module 208. The microprocessor 122 of the insufflator 202 on reading the signals indicative of the maximum and minimum safe working pressure values from the first communications module 208 stores the maximum and minimum safe working pressure values in the memory 145.

Once the first and second transition pressure values have been determined and the graphical representation of the first pressure/volume relationship has been prepared, and displayed on the touch screen 217 of the controller 205, and the maximum and minimum safe working pressure values have been stored in the memories 145 and 214 of the insufflator 202 and the controller 205, respectively, the insufflator 202 is then ready to operate in the normal insufflating run mode for insufflating the cavity 3 to a desired working pressure value. The surgeon selects the appropriate desired working pressure value based on the first and second transition pressure values displayed on the touch screen 217 and on the visual display screen 228 of the controller 205. The surgeon enters the desired working pressure value into the microprocessor 212 through the touch screen 217 of the controller 205. The desired working pressure value may be entered through a key pad displayed on the touch screen 217 or may be selected from the graphical representation of the first pressure/volume relationship displayed on the touch screen 217 by touching the pressure value on the graphical representation of the first pressure/volume relationship corresponding to the desired working pressure value, or by placing a cursor on the pressure value on the graphical representation of the first pressure/volume relationship corresponding to the desired working pressure value. Alternatively, the surgeon may select the desired working cavity volume from the graphical representation of the first pressure/volume relationship displayed on the touch screen 217 of the controller by touching the graphical representation of the first pressure/volume relationship displayed on the touchscreen 217 at the cavity volume corresponding to the desired working cavity volume or by placing a cursor on the corresponding cavity volume on the first pressure/volume relationship.

The microprocessor 212 operates the second communications module 209 to transmit a signal indicative of the selected desired working pressure value or the desired working cavity volume for reception by the first communications module 208 of the insufflator 202. The microprocessor 122 of the insufflator 202 on reading the signal indicative of the selected desired working pressure value or the selected desired working cavity volume from the first communications module 208 stores the selected desired working pressure value or the selected desired working cavity volume in the memory 145.

The surgeon then selects the normal insufflating run mode of the insufflator 202 by operating the normal insufflating run mode select button operated switch 220 of the controller 205. On the microprocessor 212 detecting the signal from the normal insufflating run mode select button operated switch 220, the microprocessor 212 operates the second communications module 209 to transmit a signal for reception by the first communications module 208 of the insufflator 202 indicative of the selection of the normal insufflating run mode.

The microprocessor 122 of the insufflator 202 on reading the signal indicative of the selection of the normal insufflating run mode operates the flow controller 124 to commence insufflating of the peritoneal cavity 3 at the selected desired working pressure value stored in the memory 145, or at a pressure value corresponding to the selected desired working cavity volume which the microprocessor 122 determines from the stored first pressure/volume relationship stored in the memory 145. The microprocessor 122 reads the signal from the pressure sensor 137 and operates the flow controller 124 to maintain the cavity pressure in the peritoneal cavity 3 at the selected desired working pressure or the pressure value corresponding to the selected desired working cavity volume.

In the event of the signal read from the pressure sensor 137 being indicative of the cavity pressure in the peritoneal cavity reaching the maximum safe working pressure value stored in the memory 145, the microprocessor 122 operates the flow controller 124 to reduce the flow rate of insufflating gas to the cavity 3, or to pause the delivery of insufflating gas to the cavity 3 until the cavity pressure is reduced to the desired working pressure value or is reduced below the maximum safe working pressure value. If the signal read from the pressure sensor 137 by the microprocessor 122 of the insufflator 202 is indicative of failure of the pressure in the peritoneal cavity 3 to fall below the maximum safe working pressure value, the microprocessor 122 operates the solenoid operated two-way valve 127 into the second state to connect the vacuum pump 171 to the outlet port 132, and the microprocessor 122 operates the vacuum pump 171 until the pressure in the peritoneal cavity 3 returns to the selected desired working pressure value or to the pressure value corresponding to the selected desired cavity volume. At which stage, the solenoid operated two-way valve 127 is operated into the first state to connect the flow controller 124 to the outlet port 132.

If the cavity pressure still fails to fall below the maximum safe working pressure value, the microprocessor 122 operates the alarm sounder 148 and the warning light 149 of the insufflator 202 to produce a human sensory perceptible signal, namely, an audible warning signal and a warning light signal indicative of the cavity pressure reaching the maximum safe working pressure value. Additionally, the microprocessor 122 operates the first communications module 208 to transmit a signal indicative of the cavity pressure in the peritoneal cavity 3 failing to fall below the maximum safe working pressure value for reception by the second communications module 209. The microprocessor 212 of the controller 205 on reading the signal from the second communications module 209 indicative of the pressure in the peritoneal cavity 3 failing to fall below the maximum safe working pressure value operates the alarm sounder 229 and the warning light 230 to produce a human sensory perceptible signal indicative of the cavity pressure in the peritoneal cavity 3 failing to fall below the maximum safe working pressure value, namely, an audible warning signal and a visual warning light signal to indicate to the surgeon that the pressure in the peritoneal cavity 3 has failed to fall below the maximum safe working pressure value.

In the event of the alarm sounder 229 and/or the warning light 230 producing a warning signal indicative of the pressure in the peritoneal cavity failing to fall below the maximum safe working pressure value, a control signal may be entered through the touch screen 217 to the microprocessor 212 to reduce the cavity pressure to the desired working pressure value or the pressure value corresponding to the selected desired working cavity volume. The microprocessor 212 on reading the control signal from the touch screen 217 operates the second communications module 209 to transmit a signal indicative of a request to reduce the cavity pressure to the desired working pressure value for reception by the first communications module 208 of the insufflator. The microprocessor 122 of the insufflator on reading the signal indicative of a request to reduce the cavity pressure to the desired working pressure value from the first communications module 208 operates the flow controller to decrease the rate at which insufflating gas is being delivered to the peritoneal cavity 3 or to pause delivery of insufflating gas to the cavity 3 or operate the vacuum pump 171 as the case may be in order to reduce the cavity pressure to the selected desired working pressure value. If the increase in the cavity pressure to the maximum safe working pressure value resulted from operation of the first foot pedal operated switch 222, the cavity pressure may be reduced below the maximum safe working pressure value by operating the second foot pedal operated switch 223.

On the microprocessor 122 of the insufflator 202 determining from the signal read from the pressure sensor 137 that the pressure in the peritoneal cavity 3 has fallen to the minimum safe working pressure value, the microprocessor 122 operates the flow controller 124 to increase the flow rate of insufflating gas to the peritoneal cavity 3 to increase the cavity pressure in the peritoneal cavity 3 to the selected desired working pressure value or the pressure value corresponding to the selected desired working cavity volume. If the flow controller 124 fails to increase the cavity pressure above the minimum safe working pressure value, the microprocessor 122 operates the first communications module 208 to transmit a signal indicative of the pressure in the peritoneal cavity 3 being at the minimum safe working pressure value for reception by the second communications module 209 of the controller 205. The microprocessor 212 of the controller 205 on reading the signal indicative of the cavity pressure being at the minimum safe working pressure value from the second communications module 209 operates the alarm sounder 229 and the warning light 230 to produce a human sensory perceptible signal indicative of the pressure in the peritoneal cavity 3 being at or below the minimum safe working pressure value, namely, an audible warning signal and a visual warning light signal.

In the event of the alarm sounder 229 and/or the warning light 230 producing a warning signal indicative of the pressure in the peritoneal cavity 3 being at or below the minimum safe working pressure value, a control signal may be entered through the touch screen 217 of the controller 205 to the microprocessor 212 thereof to operate the flow controller 124 at its maximum flow rate. On receiving this control signal the microprocessor 212 of the controller 205 operates the second communications module 209 to transmit a signal instructing the flow controller 124 to be operated at its maximum flow rate. The microprocessor 122 of the insufflator 202 on reading the signal instructing the flow controller 124 to be operated at its maximum flow rate from the first communications module 208 operates the flow controller 124 to deliver insufflating gas at its maximum flow rate to the peritoneal cavity 3.

If the reduction in the cavity pressure to the minimum safe working pressure value is as a result of operation of the second foot pedal operated switch 223, the cavity pressure may be increased above the minimum safe working pressure value by operating the first foot pedal operated switch 222.

While the microprocessor 122 of the insufflator 202 has been described as determining the pressure/volume relationship between the cavity pressure and the volume of the cavity 3 from the signals read from the flow sensor 135 and from the pressure sensor 137 when the insufflator 202 is operating in the set-up mode, it is envisaged that in some embodiments of the invention the microprocessor 212 of the controller 205 may be programmed to determine the pressure/volume relationship between the cavity pressure and the volume of the peritoneal cavity 3. In which case, it is envisaged that the microprocessor 122 of the insufflator would operate the first communications module 208 to transmit the signals read from the flow sensor 135 and from the pressure sensor 137 at the predefined sampling time intervals of 1 second to 1.5 seconds from commencement of insufflating of the cavity 3 in the set-up mode for reception by the second communications module 209 of the controller 205.

The microprocessor 212 of the controller 205 would determine the pressure/volume relationship between the cavity pressure and the volume of the peritoneal cavity 3 from the signals read from the second communications module 209. The microprocessor 212 of the controller 205 would then determine the first and second transition pressure values from the pressure/volume relationship, and would prepare the graphical representation of the first pressure/volume relationship from the first transition pressure value to the second transition pressure value for the peritoneal cavity 3. The first and second transition pressure values and a digital representation of the first pressure/volume relationship for the peritoneal cavity 3 would be stored in the memory 214 of the controller 215. The microprocessor 212 would then display the graphical representation of the first pressure/volume relationship from the first transition pressure value to the second transition pressure value on the touch screen 217 of the controller 205, and would display the first and second transition pressure values on the visual display screen 228. The microprocessor 212 of the controller 205 may then be programmed to transmit signals indicative of the first and second transition pressure values and the graphical representation of the first pressure/volume relationship through the second communications module 209 for reception by the first communications module 208 of the insufflator 202. On the microprocessor 122 reading the signals from the first communications module 208 the microprocessor 122 would then store the signal indicative of the first and second transition pressure values and the digital representation of the first pressure/volume relationship in the memory 145 of the insufflator 202 for the peritoneal cavity 3.

It is also envisaged that instead of the surgeon initiating the insufflator 202 to operate in the set-up mode, a nurse or a clinician in the first location may operate the insufflator 202 to operate in the set-up mode by operating the set-up mode select button operated switch 142 in the interface 140 of the insufflator 202.

Instead of or as well as the microprocessor 122 of the insufflator 202 or the microprocessor 212 of the controller 205 producing the graphical representation of the first pressure/volume relationship for the peritoneal cavity 3 when operating in the set-up mode, it is envisaged that either of the microprocessor 122 of the insufflator 202 or the microprocessor 212 of the controller 205 may be programmed to compute a look-up table similar to the look-up table 160 of FIG. 5 corresponding to the first pressure/volume relationship for the peritoneal cavity 3, the look-up table 160 would be displayed on the touch screen 217 of the interface 215 of the controller 205 and on the touch screen 141 of the interface 140 of the insufflator 202. A digital data representation of the look-up table 160 would be stored in the memory 145 of the insufflator 202 and in the memory 214 of the controller 205. The digital data representation of the look-up table 160 would be transmitted by the insufflator 202 to the controller 205, or vice versa, depending on which of the microprocessors 122 or 212 produce the look-up table 160, and the digital data representation of the look-up table 160. The surgeon in the second location would then select the desired working pressure value or the desired working cavity volume from the touch screen 217 of the interface 215 of the controller 205 by touching the pressure value or the cavity volume in the look-up table or by selecting the pressure value or the cavity volume in the look-up table by a cursor corresponding to the desired working pressure value or the desired working cavity volume.

Additionally, it is envisaged that in some embodiments of the invention the maximum safe working pressure value and the minimum safe working pressure value may be determined by either of the microprocessors 122 or 212 of the insufflator 202 or the controller 205, respectively. If the maximum safe working pressure value and the minimum safe working pressure value is determined by either of the microprocessors 122 or 212, it is envisaged that the maximum safe working pressure value would be determined as the second transition pressure value, namely, the pressure value at which the first pressure/volume relationship transitions to the second pressure/volume relationship, and the minimum safe working pressure value would be determined as a pressure value of between 2 mmHg and 5 mmHg above the first transition pressure value, namely, the pressure value at which the initial pressure/volume relationship transitions to the first pressure/volume relationship.

On the maximum and minimum safe working pressure values being determined by either the microprocessor 122 of the insufflator 202 or the microprocessor 212 of the controller 205, the maximum and minimum safe working pressure values are stored in the memory 145 of the insufflator 202 and the memory 214 of the controller 205 and are displayed on the visual display screens 147 and 228 of the insufflator 202 and the controller 205, respectively. It will be understood by those skilled in the art that the microprocessor 122 or 212 that determines the maximum and minimum safe working pressure values will communicate the determined values of the maximum and minimum safe working pressure values to the other one of the microprocessors 122 and 212 through the first and second communications modules 208 and 209.

In use, with the subject 5 and the insufflator 202 in the first location 203, and the insufflator 202 connected through the connecting conduit 134 and the trocar 106a to the peritoneal cavity 3 of the subject 5, and with the controller 205 in the second location 206 and communicating with the insufflator 202 through the first and second communications modules 208 and 209, the insufflating system 200 is ready for use. Initially, the surgeon requests a nurse or a clinician in the first location 203 to activate the insufflator 202. With the insufflator 202 activated, the surgeon by operating the set-up select button operated switch 219 of the controller 205 activates the insufflator 202 to operate in the set-up mode in order to determine the pressure/volume relationship between the pressure in the peritoneal cavity 3 of the subject 5 and the volume of the peritoneal cavity 3, and in turn to determine the first pressure/volume relationship between the pressure in the peritoneal cavity 3 and the volume of the peritoneal cavity 3. Alternatively, the surgeon may request the nurse or clinician in the first location to operate the insufflator 202 in the set-up mode by operating the set-up select button operated switch 142 of the insufflator 202.

With the first pressure/volume relationship of the pressure/volume relationship between the pressure in the peritoneal cavity 3 and the volume of the peritoneal cavity 3 determined, digital data representative of the first pressure/volume relationship is stored in the memories 145 and 214. The microprocessor 122 of the insufflator 202 determines the first transition pressure value and the second transition pressure value from the first pressure/volume relationship between the cavity pressure and the cavity volume of the peritoneal cavity 3 and stores the first and second transition pressure values in the memories 145 and 214. The first transition pressure value is determined as the pressure value at which the initial pressure/volume relationship transitions to the first pressure/volume relationship, and the second transition pressure value is determined as the pressure value at which the first pressure/volume relationship transitions to the second pressure/volume relationship.

With the first and second transition pressure values determined, the first and second transition pressure values are displayed on the visual display screen 147 and the touch screen 141 of the insufflator 202 and on the visual display screen 228 and the touch screen 217 of the controller 205.

If either of the microprocessors 122 or 212 are programmed to produce the graphical representation of the first pressure/volume relationship, the graphical representation of the first pressure/volume relationship from the first transition pressure value to the second transition pressure value is displayed on the touch screens 141 and 217 of the insufflator 202 and the controller 205, respectively. Alternatively, if either of the microprocessors 122 or 212 are programmed to produce a look-up table similar to the look-up table 160 of FIG. 5, the look-up table 160 is displayed on the touch screen 141 and the touch screen 217 of the insufflator 202 and the controller 205, respectively.

If the graphical representation of the first pressure/volume relationship is displayed on the touch screen 141 and 217, the surgeon in the second location may select the desired working pressure value at which the peritoneal cavity 3 is to be insufflated or the desired working cavity volume at which the peritoneal cavity 3 is to be insufflated by either touching the graphical representation of the first pressure/volume relationship on the touch screen 217 at the pressure value or the cavity volume corresponding to the desired working pressure value or the desired working cavity volume. Alternatively, if the touch screen 217 is set up with a moveable cursor, the surgeon in the second location selects the desired working pressure value or the desired working cavity volume by placing the cursor on the graphical representation of the first pressure/volume relationship at the pressure value or the cavity volume corresponding to the desired working pressure value or the desired working cavity volume and selecting that pressure value or cavity volume by the cursor.

If on the other hand, instead of displaying the graphical representation of the first pressure/volume relationship, a look-up table similar to the look-up table 160 is displayed on the touch screen 217 of the controller 205, the surgeon selects the desired working pressure value or the desired working cavity volume from the look-up table 160 displayed on the touch screen 217 by touching the value of the cavity pressure or the cavity volume corresponding to the desired working pressure value or the desired working cavity volume in the look-up table, or by selecting corresponding cavity pressure or cavity volume from the look-up table by a cursor. If neither of the microprocessors 122 or 212 are programmed to determine the maximum safe working pressure value and the minimum safe working pressure value, the surgeon in the second location inputs the maximum safe working pressure value and the minimum safe working pressure value through the touch screen 217 of the controller 205. The maximum and minimum safe working pressure values are entered through the touch screen 217 by selecting the pressure values corresponding to the maximum and minimum safe working pressure values from the graphical representation of the first pressure/volume relationship or from the look-up table, as the case may be, by touching the relevant pressure values or by selecting the relevant pressure values by a cursor on the touch screen 217. The selected maximum and minimum safe working pressure values are stored in the memory 214 of the controller 205, and are transmitted through the first and second communications modules 208 and 209 under the control of the microprocessor 212 to the microprocessor 122 and are stored in the memory 145 of the insufflator 202.

With the desired working pressure value or the desired working cavity volume selected and stored in the memories 145 and 214 of the insufflator 202 and the controller 205, respectively, and with the maximum and minimum safe working pressure values also stored in the memories 145 and 214, the insufflator 202 is ready to operate in the normal insufflating run mode. The surgeon in the second location operates the normal insufflating run mode select button operated switch 220 in the interface 215 of the controller 205 to operate the insufflator 202 in the normal insufflating run mode. If the solenoid operated two-way valve 127 is not set in the first state connecting the flow controller 124 to the outlet port 132, the microprocessor 122 sets the solenoid operated two-way valve 127 in the first state with the flow controller 124 connected through to the outlet port 132. The microprocessor 122 operates the flow controller 124 to insufflate the cavity 3 and reads the signal from the pressure sensor 137 and controls the flow controller 124 to supply the insufflating gas to the cavity 3 at a suitable flow rate to maintain the cavity pressure or the cavity volume at the selected desired working pressure value or the selected desired working cavity volume.

If during insufflating of the cavity 3, the surgeon in the second location wishes to increase or decrease the cavity pressure, the surgeon operates the appropriate one of the first and second pedal operated switches 222 or 223 of the controller 205 in a similar manner as the foot pedal operated switches 172 and 173 of the insufflator 170 of FIG. 6 are operated. As already described with reference to the insufflator 170 of FIG. 6, each operation of the foot pedal switch 222 increases the cavity pressure by a single increment, and each operation of the second foot pedal operated switch 223 decreases the cavity pressure by a single decrement. Thus, the microprocessor 212 of the controller 205 counts the number of operations of the first foot pedal operated switch 222 or the second foot pedal operated switch 223 as the case may be, and determines the number of increments or decrements by which the cavity pressure should be increased or decreased from the selected desired working pressure or from the pressure value corresponding to the selected desired working cavity volume. A signal indicative of the number of increments by which the cavity pressure is to be increased or a signal indicative of the number of decrements by which the cavity pressure is to be decreased is transmitted by the microprocessor 212 of the controller 205 to the microprocessor 122 of the insufflator 202 through the second and first communication modules 209 and 208. The microprocessor 122 of the insufflator 202 on reading the signal from the first communications module 208 then stores the new value of the pressure to which the cavity pressure is to be altered in the memory 145, and operates either the flow controller 124 or the flow controller 124 and the vacuum pump 171 of the insufflator 202 and also operates the solenoid operated two-way valve 127 in the appropriate one of the first and second states thereof, as already described with reference to the insufflator 170 of FIG. 6 to alter the cavity pressure to the desired altered cavity pressure value.

The value of each increment or decrement by which the cavity pressure is to be altered may be, as discussed with reference to the insufflator 170 of FIG. 6, 5% of the optimum maximum pressure value Pₘₐₓ which in this embodiment of the invention is 5% of the maximum safe working pressure value. Otherwise, the value of each increment or decrement may be pre-entered into the memory 214 of the controller 205 by the surgeon in the second location through the touch screen 217 of the controller 205, and transmitted through the second and first communications module 209 and 208 for storing in the memory 145 of the insufflator 202.

It is envisaged that the increments or decrements by which the cavity pressure may be increased or decreased by operating the first or second foot pedal operated switches 222 or 223 may be greater than or less than 5% of the maximum safe working pressure value. For example, in some embodiments of the invention the increments or decrements may be of the order of as low as 2% of the maximum safe working pressure value or even lower than 2% thereof, or may be as high as 10% of the maximum safe working pressure value and even higher than 10% thereof.

If during insufflating of the peritoneal cavity 3 of the subject 5 in the set-up mode, or in the normal insufflating run mode, the cavity pressure reaches the maximum safe working pressure value or fails to fall below the maximum safe working pressure value, the microprocessor 122 of the insufflator 202 operates the alarm sounder 148 and the warning light 149 of the insufflator 202 to produce a corresponding warning signal, and the microprocessor 122 also operates the first communications module 208 to transmit a corresponding signal for reception by the second communications module 209 of the controller 205. On the microprocessor 212 of the controller 205 reading the signal from the second communications module 209, the microprocessor 212 operates the alarm sounder 229 and the warning light 230 of the controller 205 to produce the corresponding warning signal to the surgeon in the second location. Either the surgeon in the second location, or the nurse or clinician in the first location on being alerted by the signal produced by the relevant alarm sounder 148 and 229 and the relevant warning light 149 and 230, may then take appropriate action to reduce the cavity pressure in the peritoneal cavity below the maximum safe working pressure value. Such action which may be taken by either the nurse or clinician in the first location or the surgeon in the second location, would be to input the appropriate signal through the touch screen 141 or the touch screen 217 to operate the insufflator 202 to reduce the cavity pressure below the maximum safe working pressure value.

On the other hand, should the microprocessor 122 determine from the signal read from the pressure sensor 137 that the cavity pressure in the peritoneal cavity 3 has fallen to the minimum safe working pressure value or fails to rise above the minimum safe working pressure value, the microprocessor 122 operates the alarm sounder 148 and the warning light 149 to produce a corresponding signal indicative of the cavity pressure having fallen to the minimum safe working pressure value or failing to rise above the minimum safe working pressure value. The microprocessor 122 also operates the first communications module 208 to transmit a signal for reception by the second communications module 209 of the controller 205 indicative of the cavity pressure having fallen to the minimum safe working pressure value or failing to rise above the minimum safe working pressure value. On the microprocessor 212 of the controller 205 reading the signal from the second communications module 209 indicative of the cavity pressure having fallen to the minimum safe working pressure value or failing to rise above the minimum safe working pressure value, the microprocessor 212 operates the alarm sounder 229 and the warning light 230 to produce an appropriate warning signal indicative of the cavity pressure having fallen to the minimum safe working pressure value or failing to rise above the minimum safe working pressure value. Either the nurse or clinician in the first location or the surgeon in the second location may then input the appropriate signal through the touch screen 141 or the touch screen 217 to operate the insufflator 202 to increase the cavity pressure in the peritoneal cavity 3 above the minimum safe working pressure value or to operate the flow controller to supply the insufflating gas to the cavity 3 at the maximum flow rate.

On completion of the minimally invasive procedure, the surgeon enters a signal through the touch screen 217 to the microprocessor 212 indicative of the completion of the minimally invasive procedure, which in turn operates the second communications module 209 to transmit a corresponding signal to the first communications module 208. The microprocessor 122 of the insufflator 202 on reading the signal indicative of the completion of the minimally invasive procedure operates the solenoid operated two-way valve 127 into the second state to connect the outlet port 132 to the vacuum pump 171 and operates the vacuum pump 171 to draw insufflating gas from the peritoneal cavity 3 in order to evacuate the peritoneal cavity. Alternatively, the insufflator 202 may be operated to evacuate the peritoneal cavity by again operating the normal insufflating run mode select button operated switch 220 in the controller 205 to indicate that insufflating of the cavity 3 is to be terminated.

Accordingly, the controller 205 of the insufflating system 200 enables a surgeon to control the insufflator 202 of the insufflating system 200 for insufflating the peritoneal cavity 3 of the subject 5 as that surgeon would control the insufflator 202 if the surgeon were located in the first location 203.

Otherwise, the insufflator 202 of the insufflating system 200 is similar to the insufflator 170 and its use in insufflating a peritoneal cavity in a subject 5 is similar to that described with reference to the insufflator 170 of FIG. 6.

It is envisaged in some embodiments of the insufflator 202 that the vacuum pump 171 of the insufflator 202 may be omitted, and the insufflator would be configured for coupling to an external vacuum system, for example, a vacuum system typically of the type which is available in an operating theatre of a hospital. In which case, the vacuum pump 171 in the insufflator 202 would be replaced with an isolating valve, similar to the isolating valve described in the insufflator 100 which is described with reference to FIGS. 3 to 5. The isolating valve would be connected between a vacuum port similar to the vacuum port 120 of the insufflator 100 for connecting the insufflator 202 to a vacuum system such as the vacuum system 119 and the second inlet port 129 of the solenoid operated two-way valve 127.

It is also envisaged that while the insufflating system 200 has been described for insufflating a peritoneal cavity in the body of a human or animal subject, the insufflating system 200 may be used for insufflating any lumen, cavity or organ in the body of a human or animal subject. Additionally, while the insufflating system 200 has been described for insufflating the peritoneal cavity through a trocar in a laparoscopic minimally invasive procedure, it is envisaged that in some embodiments of the invention the insufflating system 200 may be used for insufflating any cavity, lumen, vessel or organ in a human or animal subject during the carrying out of a minimally invasive procedure endoscopically. In which case, it is envisaged that the cavity, lumen, vessel or organ being insufflated during the endoscopic minimally invasive procedure would be insufflated through an insufflating or other suitable channel of an endoscope, or through a tube attached externally to and extending along the endoscope, with the tube terminating adjacent the distal end of the endoscope.

While the first and second indicating means have been described as comprising cursors, any other suitable first and second indicating means for indicating and selecting the cavity pressure to be selected and the selected working pressure value, as well as the cavity volume and the selected working cavity volume may be provided. For example, in some embodiments of the invention it is envisaged that one of the first and second cursors may comprise an arrow head, and the other of the first and second cursors may comprise a pointing finger of a hand, or each of the first and second cursors may comprise an arrow head or a pointing finger of a hand. Needless to say, any other suitable type cursor suitable as an indicating means may be provided.

It will also be appreciated that as well as the desired working pressure value being selectable from the graphical representation of the first pressure/volume relationship, and from the look-up table of the first pressure/volume relationship, the desired working cavity volume may also be selectable from the graphical representation of the first pressure/volume relationship and from the look-up table of the first pressure/volume relationship.

While in the embodiments of the invention described with reference to FIGS. 3 to 6, the optimum maximum cavity pressure value has been described as being the cavity pressure at the second point of inflection of the smoothed graph 153 of FIG. 4 representing the transition of the first pressure/volume relationship to the second pressure/volume relationship, it is envisaged that the optimum maximum pressure value may be determined as being a pressure anywhere on the graph 150 of FIG. 4 between the first pressure value P₁ at point B and the second pressure value P₂ at point C, and including the first pressure value P₁ and the second pressure value P₂, and may be a pressure closer to the first pressure value P₁ at point B on the graph 150 than to the second pressure value P₂ at point C.

While the optimum maximum pressure value has been described as being the cavity pressure at the point of inflection between the portions of the smoothed graph representing the first and second pressure/volume relationships, it is envisaged that the optimum maximum pressure value may be determined as being a pressure anywhere on the non-smoothed graph between the first pressure value P₁ at point B and the second pressure value P₂ at point C, and may be a pressure closer to the first pressure value P₁ at point B than to the second pressure value P₂ at point C.

Needless to say, other methods for determining the point of inflection of the non-smoothed graph or the points at which the transitions from the first pressure/volume relationship to the intermediate pressure/volume relationship, or from the intermediate pressure/volume relationship to the second pressure/volume relationship occur, may be used besides those described, as may other methods be used for determining the first point of inflection of the pressure/volume relationship where the initial pressure/volume relationship transitions to the first pressure/volume relationship.

While the pressure/volume relationship between the pressure in the cavity and the cumulative volume of insufflating gas delivered to the cavity has been described as comprising an initial pressure/volume relationship and first and second pressure/volume relationships, in which the slopes of the lines representing the initial pressure/volume relationship and the first and second pressure/volume relationships are substantially constant, and in which the value of the slope of the second pressure/volume relationship is greater than the value of the slope of the first pressure/volume relationship, and while the pressure/volume relationship between the first and second pressure/volume relationships is a non-linear intermediate relationship, it will be appreciated by those skilled in the art that the pressure/volume relationship between the pressure in the cavity and the corresponding cumulative volume of insufflating gas delivered to the cavity, may be other than such a pressure/volume relationship. For example, in some embodiments of the invention it is envisaged that the pressure/volume relationship may be in the form of an equation, such as a quadratic equation or other equation, which, for example, may be a power law equation. In which case, the line of the equation would be determined by, for example, curve fitting, and the slope of the line of the equation would be monitored in order to determine the point of inflection of the line at which the pressure/volume relationship transitions from the first pressure/volume relationship to the second pressure/volume relationship, and the slope of the line of the equation would also be monitored to determine the point of inflection at which the initial pressure/volume relationship transitions to the first pressure/volume relationship.

While the methods and the insufflators and the controller have been described for use in insufflating the peritoneal cavity of a subject, it will be readily apparent to those skilled in the art that the methods and insufflators and the controller may be used for insufflating any cavity, lumen or vessel in the human or animal body, and for determining the optimum maximum pressure value for any cavity, lumen or vessel in the body of a human or animal subject.

It will also be appreciated that other suitable insufflating gases besides carbon dioxide may be used.

While the flow sensors of the insufflators may be configured to produce a signal indicative of the cumulative volume of the insufflating gas delivered to the cavity, or may be configured to produce a signal indicative of the flow rate of the insufflating gas being delivered to the cavity, in either case, it is envisaged that the relevant microprocessors would be appropriately programmed to read the signals from the flow sensors at predefined sampling time intervals, typically, of 10 milliseconds each, and would be programmed to determine the appropriate pressure/volume relationship, and the values of the increase in the pressure in the cavity per unit volume of insufflating gas delivered to the cavity.

While the pressure regulator of the embodiment of the invention described with reference to FIGS. 1 and 2 has been described as stepping down the pressure of the insufflating gas to 40 mmHg, it will be appreciated by those skilled in the art that the pressure regulator may step the pressure of the insufflating gas down to any suitable pressure, and in some embodiments of the invention the pressure to which the insufflating gas is stepped down by the pressure regulator may be less than or greater than 40 mmHg. Indeed, in some embodiments of the invention the pressure regulator may be configured to step down the pressure of the insufflating gas to a pressure significantly higher than the pressure of 40 mmHg, and in some cases, may only reduce the pressure of the insufflating gas to a pressure in the order of 3.5 bar. In which case, the flow controller would be configured to reduce the pressure from such a pressure to a suitable pressure, such that the insufflating gas would be supplied to the cavity at the appropriate pressure, such that during the procedure, the pressure in the cavity would be maintained at the selected working pressure, or at any other pressure chosen or selected by the surgeon or clinician. During initial insufflating of the cavity in order to determine the set pressure value, the flow controller would be operated to supply the insufflating gas to the cavity at the appropriate constant rate.

While the pressure/volume relationship between the cavity pressure and the cumulative volume of insufflating gas delivered to the cavity has been described as comprising first and second pressure/volume relationships, in which the slopes of the respective portions representing the first and second pressure/volume relationships are substantially constant, and in which the value of the slope of the second pressure/volume relationship is greater than the value of the slope of the first pressure/volume relationship, and while the intermediate pressure/volume relationship between the first and second pressure/volume relationships is a non-linear intermediate relationship, it will be appreciated by those skilled in the art that the pressure/volume relationship between the cavity pressure and the corresponding cumulative volume of insufflating gas delivered to the cavity, may be other than such a pressure/volume relationship. For example, in some embodiments of the invention it is envisaged that the pressure/volume relationship of the working pressure range from the minimum pressure value or the first pressure value to the optimum maximum cavity pressure value or the second pressure value may be a non-linear relationship, and in which case the pressure/volume relationship may be stored in memory in the form of an equation, such as a quadratic equation or other suitable equation, which, for example, may be a power law equation. In which case, the equation would be determined by, for example, curve fitting, and the slope of the portion of the equation representing the working pressure range would be monitored in order to determine the points of inflection of the equation which would determine the minimum pressure value or the first pressure value and the optimum maximum cavity pressure value or the second pressure value, which in turn would determine the working pressure range of the pressure/volume relationship.

While the interface means has been described as comprising a touch screen interface, and set-up mode and normal insufflating run mode select button operated switches, any other suitable interface means may be provided, for example, a graphic user interface screen, a keypad, and in some embodiments of the invention the insufflators of FIGS. 1 to 8 may be operated remotely, for example, wirelessly from a smart mobile device, for example, a smart mobile phone, or by a button operated switch or other suitable interface means on the camera head of a laparoscope, an endoscope or a colonoscope.

It is also envisaged that the secondary input means or the interface means may include other suitable means for selecting the desired working pressure value or the desired working cavity volume to which the cavity is to be insufflated, and such means may include the provision of, for example, manually operated switches, whereby one manually operated switch would be provided to increase the working pressure value or the working cavity volume of the cavity, and one manually operated switch would be provided to reduce the working pressure value or the working cavity volume of the cavity. The manual switches may be configured such that for each operation of the relevant switch one step change in the working pressure value or the working cavity volume of the cavity would occur, or the switches may be configured such that for so long as the relevant switch remains activated, the working pressure value or the working cavity volume of the cavity would continue to be increased or decreased as the case may be. It is also envisaged that the foot pedal operated switches of the insufflator of FIGS. 6 and 8 may be configured such that for so long as the relevant foot pedal switch remains in the closed circuit state, the microprocessor would operate the flow controller, the isolating valve or the vacuum pump, as the case may be, to continue to increase or decrease the cavity pressure until the cavity pressure or the volume of the cavity was at the selected or desired working pressure value or the selected or desired working cavity volume. It is also envisaged that the manually or foot pedal operated switches may comprise analogue switches, such as rheostat switches, or may comprise digital switches, which would produce an analogue type output signal proportional to the degree to which the switch is operated from the open circuit state, and the degree to which the cavity pressure would be increased or decreased would be proportional to the degree to which the relevant switch is operated from the open circuit state.

It is envisaged that in some embodiments of the invention the insufflators may be configured to operate in the normal insufflating run mode only. In which case, it is envisaged that one or more look-up tables would be stored electronically in the memory of the insufflator, the look-up tables would be similar to the look-up table described with reference to the insufflator of FIGS. 3 to 5, and each look-up table would store the selectable working pressure values and may also store selectable working cavity volume and the corresponding cavity pressures as illustrated in the look-up table of FIG. 5. The look-up tables would store the selectable working pressure values and selectable working cavity volume for different types of cavities of the human or animal body, and it is also envisaged that a number of look-up tables would be provided for each different type of cavity, lumen, vessel or organ for humans or animals of different sex, different ages, different body mass indices and other relevant biometric data. It is also envisaged that instead of such look-up tables the working pressure values and the corresponding working cavity volumes, and other data may be stored in memory in the form of one or more equations indicative of the relevant pressure/volume relationships for the working pressure ranges, or as a series of graphical representations of pressure/volume relationships for different types of cavities, lumens, vessels or organs for subjects of different sex, different ages, different body mass indices and other relevant biometric data.

While the isolating valve of the insufflator of FIGS. 3 to 5 and the vacuum pumps of FIGS. 6 and 8 have been described as being connected to the outlet port 132 through the solenoid operated two-way valve 127, it is envisaged that the isolating valve or the vacuum pumps may be connected directly to a separate outlet port in the housing, and a separate conduit would be provided from that outlet port to the cavity 3 through either one of the trocars 106a or 106b or through a separate trocar. In cases where the isolating valve or the vacuum pump is connected directly to a separate outlet port, the solenoid operated two-way valve 127 may be omitted and the flow controller would be connected to the outlet port 132. Indeed, in some embodiments of the invention, it is envisaged that instead of the isolating valve being provided to form a part of the flow control means, the isolating valve may be omitted and the two-way valve 127 would act in place of the isolating valve for selectively connecting the vacuum system to the cavity.

It is also envisaged that the insufflators according to the invention may include provision for monitoring leakage from the cavity being insufflated, and compensating values would be provided to compensate for leakage, both during operation of the insufflator in the set-up mode and during operation of the insufflator in the normal insufflating run mode.

While the look-up table has been described as comprising the selectable working cavity volumes as percentages of the maximum cavity volume or of the maximum safe working cavity volume, in some embodiments of the invention it is envisaged that the look-up table may comprise specific values of the selectable working cavity volumes of the cavity.

It is also envisaged that the selectable working pressure values and/or the selectable working cavity volume may be of any values, and while they have been described in the look-up table as being provided as incremental values of 5% of the maximum cavity volume or 5% of the maximum safe working cavity volume, and 5% of the optimum maximum pressure value or 5% of the maximum safe working cavity volume, the incremental values may be of any suitable values, and the values of the incremental values will largely be determined by the number of selectable pressure or volume values which will be provided within the working pressure range and the working volume range.

While the insufflators and the methods as well as the insufflating system and the methods have been described for use in a laparoscopic procedure, it is envisaged that the insufflators and the method, as well as the insufflating system and the method may be used in any minimally invasive procedure, such as a minimally invasive procedure carried out by an endoscope, a colonoscope, or indeed any other suitable instrument for carrying out a minimally invasive procedure. Such minimally invasive procedures as discussed above, may be minimally invasive surgical procedures or minimally invasive investigative procedures.

1. A method for remotely insufflating a cavity in the body of a human or an animal subject,
the subject being in a first location,
an insufflator located in the first location and connected to the cavity of the subject for insufflating thereof, the insufflator comprising a first communicating means, and
a controller located in a second location remote from the first location for controlling operation of the insufflator, the controller comprising a second communicating means adapted to remotely communicate with the first communicating means of the insufflator, for communicating control signals from the controller to the insufflator for controlling the operation of the insufflator, the method comprising:
operating the controller to transmit a signal indicative of a desired working pressure value at which the cavity of the subject is to be maintained during insufflating thereof for reception by the first communicating means of the insufflator, and
operating the controller to transmit a signal for reception by the first communicating means of the insufflator to operate the insufflator in an insufflating mode to insufflate the cavity of the subject at the desired working pressure value,
the desired working pressure value being a pressure value selectable from a range of pressure values between a first transition pressure value and a second transition pressure value,
the first transition pressure value being a pressure value in a pressure/volume relationship between the pressure in the cavity (cavity pressure) and the volume of the cavity of the subject at which the pressure/volume relationship transitions from an initial pressure/volume relationship during which the cavity pressure remains substantially constant while insufflating gas is being delivered to the cavity, to a first pressure/volume relationship during which the cavity pressure increases per unit volume of insufflating gas delivered to the cavity, and
the second transition pressure value being a pressure value in the pressure/volume relationship at which the first pressure/volume relationship transitions to a second pressure/volume relationship during which the cavity pressure increases per unit volume of insufflating gas delivered to the cavity at a greater rate than the cavity pressure increases per unit volume of insufflating gas delivered to the cavity during the first pressure/volume relationship.

2. A method as claimed in claim 1 in which the first and second locations are located in respective different buildings, in respective different towns or cities, or in respective different countries, and the first and second communicating means are adapted to communicate wirelessly, by wire and/or fibreoptically.

3. A method as claimed in claim 1 in which the controller comprises an interface means, and the desired working pressure value is inputted to the controller through the interface means thereof for transmission by the controller to the insufflator.

4. A method as claimed in claim 3 in which a pressure value to which the cavity pressure is to be altered from the current cavity pressure is inputted through the interface means of the controller, and the controller is adapted to transmit to the insufflator an alter cavity pressure request signal indicative of the pressure value to which the cavity pressure is to be altered from the current cavity pressure during insufflating of the cavity.

5. A method as claimed in claim 4 in which the interface means of the controller comprises at least one switch means, and the at least one switch means is adapted for inputting the pressure value to which the cavity pressure is to be altered from the current cavity pressure and for producing the alter cavity pressure request signal for transmission to the insufflator.

6. A method as claimed in claim 5 in which the interface means of the controller comprises a pair of the switch means, a first one of the switch means being adapted for inputting a pressure value to which the cavity pressure is to be increased from the current cavity pressure and for producing the alter cavity pressure request signal as an increase pressure request signal indicative of the pressure value to which the cavity pressure is to be increased from the current cavity pressure, and a second one of the switch means being adapted for inputting a pressure value to which the cavity pressure is to be reduced from the current cavity pressure and for producing the alter cavity pressure request signal as a decrease pressure request signal indicative of the pressure value to which the cavity pressure is to be reduced from the current cavity pressure.

7. A method as claimed in claim 6 in which each switch means of the controller comprises a foot pedal operated switch.

8. A method as claimed in claim 1 in which the first and second transition pressure values are visually displayed on the interface means of the controller.

9. A method as claimed in claim 1 in which the first pressure/volume relationship is visually displayed on the interface means of the controller as a graphical representation thereof.

10. A method as claimed in claim 9 in which the graphical representation of the first pressure/volume relationship displayed on the interface means of the controller is displayed in the form of a graph, with the cavity pressure plotted on the ordinate or the abscissa, and the cavity volume being plotted on the other one of the ordinate and the abscissa, and the interface means is adapted to permit the desired working pressure value or a desired working cavity volume of the cavity to be inputted to the controller through the interface means for transmission to the insufflator by touching the graph at a point thereon corresponding to the pressure value or the cavity volume corresponding to the desired working pressure value or the desired working cavity volume or by positioning a cursor on the graph at the pressure value or cavity volume corresponding to the desired working pressure value or the desired working cavity volume.

11. A method as claimed in claim 1 in which a look-up table is displayed on the interface means of the controller, the look-up table displaying the first transition pressure value and a first transition cavity volume of the cavity of the subject corresponding to the first transition pressure value thereof and cross-referenced with the first transition pressure value, and the second transition pressure value and a second transition cavity volume of the cavity of the subject corresponding to the second transition pressure value thereof and cross-referenced with the second transition pressure value, and a plurality of intervening pressures of values lying between the first transition pressure value and the second transition pressure value and respective intervening cavity volumes of the cavity of the subject corresponding to the respective intervening pressure values thereof and cross-referenced with the corresponding respective intervening pressure values, and the interface means of the controller is adapted to permit entry therethrough to the controller of one of the pressure values or one of the cavity volumes displayed in the look-up table as the desired working pressure value or the desired working cavity volume for transmission to the insufflator.

12. A method as claimed in claim 11 in which the interface means of the controller is adapted for entering the one of the pressure values or the one of the cavity volumes displayed in the look-up table thereof as the desired working pressure value or the desired working cavity volume to the controller by touching the pressure value or the cavity volume in the look-up table displayed in the interface means corresponding to the desired working pressure value to be selected or the desired working cavity volume to be selected, or by identifying the pressure value or the cavity volume in the look-up table displayed in the interface means by a cursor corresponding to the desired working pressure value to be selected or the desired working cavity volume to be selected.

13. A method as claimed in claim 1 in which the insufflator is operable under the control of the controller in a set-up mode, and the insufflator or the controller is operable to determine the first transition pressure value and the second transition pressure value as the insufflator is operating in the set-up mode.

14. A method as claimed in claim 13 in which the insufflator or the controller is programmed to determine the pressure/volume relationship between the cavity pressure and the volume of the cavity of the subject when the insufflator is operating in the set-up mode for determining the first transition pressure value and the second transition pressure value.

15. A method as claimed in claim 13 in which the interface means of the controller is adapted for inputting a set-up mode request, and the controller is adapted to produce a set-up mode request signal for transmission to the insufflator to operate the insufflator in the set-up mode in response to inputting of the set-up mode request.

16. A method as claimed in claim 1 in which the interface means of the controller is adapted for inputting a normal insufflating mode request, and the controller is adapted to produce a normal insufflating mode request signal in response to the normal insufflating mode request for transmission to the insufflator to operate the insufflator in a normal insufflating mode for insufflating the cavity of the subject at the selected desired working pressure value.

17. A method as claimed in claim 1 in which the interface means of the controller is adapted for inputting a minimum safe working pressure value below which the cavity pressure should not fall during insufflating thereof, and the controller is adapted to transmit a signal to the insufflator indicative of the minimum safe working pressure value in response to the inputting of the minimum safe working pressure value, and the insufflator is programmed to store the minimum safe working pressure value and to maintain the cavity pressure above the minimum safe working pressure value during insufflating of the cavity.

18. A method as claimed in claim 1 in which the interface means of the controller is adapted for inputting a maximum safe working pressure value above which the cavity pressure should not exceed during insufflating thereof, and the controller is adapted to transmit a signal to the insufflator indicative of the maximum safe working pressure value in response to the inputting of the maximum safe working pressure value, and the insufflator is programmed to store the maximum safe working pressure value and to maintain the cavity pressure below the maximum safe working pressure value during insufflating of the cavity.

19. A method as claimed in claim 1 in which the cavity in the body of the human or animal subject comprises the peritoneal cavity.

20. An insufflating system for remotely insufflating a cavity in the body of a human or animal subject, the insufflating system comprising:
an insufflator located in a first location and connected to the cavity of the human or animal subject, the insufflator comprising a first communicating means,
a controller located in a second location remote from the first location, the controller comprising a second communicating means adapted for communicating with the first communicating means of the insufflator,
the controller being adapted to control the operation of the insufflator through the first and second communicating means for insufflating the cavity of the subject at a desired working pressure value at which the cavity is to be maintained, the desired working pressure value being a pressure value selectable from a range of pressure values between a first transition pressure value and a second transition pressure value,
the first transition pressure value being a pressure value in a pressure/volume relationship between the pressure in the cavity (cavity pressure) and the volume of the cavity of the subject at which the pressure/volume relationship transitions from an initial pressure/volume relationship during which the cavity pressure remains substantially constant while insufflating gas is being delivered to the cavity to a first pressure/volume relationship during which the cavity pressure increases per unit volume of insufflating gas delivered to the cavity, and
the second transition pressure value being a pressure value in the pressure/volume relationship at which the first pressure/volume relationship transitions to a second pressure/volume relationship during which the cavity pressure increases per unit volume of insufflating gas delivered to the cavity at a greater rate than the cavity pressure increases per unit volume of insufflating gas delivered to the cavity during the first pressure/volume relationship.
